(19) United States
(12) Patent Application Publication
(10) Pub. No.: US 2012/0329115 A1
(43) Pub. Date: Dec. 27, 2012

(54) CHROMOSOMAL DNA INTEGRATION METHOD

(75) Inventors: Christine SANTOS, Richmond, CA (US); Yasuo Yoshikuni, Albany, CA (US)

(73) Assignee: Bio Architecture Lab, Inc., Berkeley, CA (US)

(21) Appl. No.: 13/333,829

(22) Filed: Dec. 21, 2011

Related U.S. Application Data
(60) Provisional application No. 61/427,077, filed on Dec. 23, 2010.

(51) Int. Cl. C12N 15/90; C12P 7/06; C12N 1/21

(52) U.S. Cl. 435/161; 435/252.33; 435/477; 435/472

(57) ABSTRACT

The present disclosure relates to methods of integrating recombinant polynucleotides into genomes of unicellular organisms. In particular, the present disclosure relates to the modified unicellular organisms that contain integrated recombinant polynucleotides in their genomes and methods for production of commodity chemicals by the use of such organisms.

### CROSS-REFERENCE TO RELATED APPLICATIONS

This application claims the benefit of U.S. Provisional Application No. 61/427,077, filed Dec. 23, 2010, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 690212000100SeqList.txt, date recorded: Dec. 20, 2011, size: 8 KB).

### FIELD

The present disclosure relates to the use of the Cre-lox recombination system to integrate recombinant polynucleotides into the genomes of unicellular organisms. In particular, the present disclosure relates to the modified unicellular organisms and methods for production of commodity chemicals using such organisms.

### BACKGROUND

Petroleum is facing declining global reserves and contributes to more than 30% of greenhouse gas emissions driving global warming. Annually 800 billion barrels of transportation fuel are consumed globally. Diesel and jet fuels account for greater than 50% of global transportation fuels.

Due to increasing petroleum costs and reliance on petrochemical feedstocks, the chemicals industry is also looking for ways to improve margin and price stability, while reducing its environmental footprint. The chemicals industry is striving to develop greener products that are more energy-, water-, and CO₂-efficient than current products.

One promising approach is the use of biofuels. Naturally-occurring enzymes may be used to break down polysaccharides from biomass into oligosaccharides or monosaccharides, which are then subsequently used to produce biofuels and other commodity chemicals. Such enzymes can be expressed in different host strains, such as Escherichia coli (E. coli), conferring these strains with the ability to break down biomass and produce biofuels and other commodity chemicals in an efficient and cost-effective manner.

However, there are limitations to the process of engineering host strains. Although the introduction of heterologous pathways into novel host strains has been facilitated by the ability to assemble and deliver genes on plasmids constructs, it is now commonly believed that the propagation and maintenance of plasmids within a cell can be a costly metabolic process (Birnbaum et al., 1991; Jones et al., 2000). To increase cell robustness and performance, it therefore becomes desirable to find ways to alleviate this additional cellular burden.

One such method is to integrate genes into the host genome, thus eliminating the need for plasmid-borne expression. However, because established techniques for genomic incorporation often rely on homologous recombination of single or double-stranded DNA fragments (Datsenko & Wanner, 2000; Yu et al., 2000, 2003), such methodologies possess inherent limitations with respect to both fragment size and efficiency of recombination.

The Cre-lox recombination system of bacteriophage P1, described by Abremski et al. (1983), Sternberg et al. (1981) and others, has been used to promote recombination in a variety of cell types. The Cre-lox system utilizes the Cre recombinase isolated from bacteriophage P1 in conjunction with the DNA sequences (termed lox sites) it recognizes. This recombination system has been effective for achieving recombination in plant cells (U.S. Pat. No. 5,658,772), animal cells (U.S. Pat. No. 4,959,317 and U.S. Pat. No. 5,801,030), and in viral vectors (Hardy et al., 1997). However, this technique has not been used to integrate large recombinant polynucleotides into unicellular organisms.

The problem of expressing heterologous genes in host cells is of particular relevance for the engineering of alginate metabolism in E. coli, which was found to require a suite of more than 35 heterologous genes (a total of 58 kb of foreign DNA) for optimal growth on alginate medium. Although delivery of these genes into E. coli was accomplished through the use of an F-based vector, plasmid retention rates were quite low.

Thus, these exists a need for a genetic technique capable of incorporating DNA fragments as large as 58 kb at high efficiencies into genomes of unicellular organisms.

### BRIEF SUMMARY

Provided are methods and organisms that meet this need.

The present disclosure relates to methods of integrating recombinant polynucleotides into genomes of unicellular organisms, such as gram-negative bacteria or E. coli, by the use of the Cre-lox recombination system. These methods can be used to integrate heterologous pathways of various sizes into unicellular organisms and produce a variety of commodity chemicals, such as ethanol, isobutanol, n-butanol, and 2-butanol. The present disclosure also relates to unicellular organisms, gram-negative bacteria, and E. coli strains containing integrated recombinant polynucleotides in their genomes.

Accordingly, one aspect of the present disclosure provides an E. coli strain containing a recombinant polynucleotide where the E. coli strain has a genome where the recombinant polynucleotide is stably integrated into the genome and where the recombinant polynucleotide contains a nucleotide sequence encoding an alginate lyase, a DEHU reductase, and an alginate transporter and where integration of the recombinant polynucleotide into the genome modifies the E. coli strain to be able to grow on alginate-containing or alginate-derived media.

Another aspect of the present disclosure provides an E. coli strain containing a recombinant polynucleotide where the E. coli strain has a genome where the recombinant polynucleotide is stably integrated into the genome and where the recombinant polynucleotide contains a nucleotide sequence encoding an endo-type cellulase, an exo-type cellulase, a β-glucosidase, and a cellulose/cellobiose transporter and where integration of the one or more heterologous genes into the genome modifies the E. coli strain to be able to grow on cellulose/cellobiose-containing media.

Another aspect of the present disclosure provides an E. coli strain containing a recombinant polynucleotide where the E. coli strain has a genome where the recombinant polynucleotide is stably integrated into the genome, is at least 11 kilobases in size, and has a nucleotide sequence encoding one or more heterologous genes. In some embodiments, the size of the polynucleotide is at least 12, 13, 14, 15, 16, 17, 18, 19, 20, 30, 40, 50, 100 kb (including all integers and decimal points in between, e.g., 1.2, 1.3, 1.4, 1.5, 5.5, 5.6, 5.7, 60, 70, etc.) in size. In some embodiments, the size of the recombinant polynucleotide is at least 11 kilobases. In some embodiments, the size of the recombinant polynucleotide is selected from: A) at least 12 kilobases; B) at least 13 kilobases; and C) at least 14 kilobases. In some embodiments, the one or more heterologous genes integrated into the genome is an alginate lyase, a DEHU reductase, and/or an alginate transporter and integration of the one or more heterologous genes into the genome modifies the E. coli strain to be able to grow on alginate-containing or alginate-derived media. In other embodiments, the one or more heterologous genes integrated into the genome is an endo-type cellulase, an exo-type cellulase, a β-glucosidase, and/or a cellulose/cellobiose transporter and integration of the one or more heterologous genes into the genome modifies the E. coli strain to be able to grow on cellulose/cellobiose-containing media.

In embodiments of any of the preceding aspects providing E. coli strains in any of their embodiments, the recombinant polynucleotide is positioned between two lox sites in the genome.

Another aspect of the present disclosure provides a gram-negative bacterial strain containing a recombinant polynucleotide where the gram-negative bacterial strain has a genome where the recombinant polynucleotide is stably integrated into the genome and where the recombinant polynucleotide contains a nucleotide sequence encoding an alginate lyase, a DEHU reductase, and an alginate transporter and where integration of the recombinant polynucleotide into the genome modifies the gram-negative bacterial strain to be able to grow on alginate-containing or alginate-derived media.

Another aspect of the present disclosure provides a gram-negative bacterial strain containing a recombinant polynucleotide where the gram-negative bacterial strain has a genome where the recombinant polynucleotide is stably integrated into the genome and where the recombinant polynucleotide contains a nucleotide sequence encoding an endo-type cellulase, an exo-type cellulase, a β-glucosidase, and a cellulose/cellobiose transporter and where integration of the one or more heterologous genes into the genome modifies the gram-negative bacterial strain to be able to grow on cellulose/cellobiose-containing media.

Yet another aspect of the present disclosure provides a unicellular organism containing a recombinant polynucleotide where the unicellular organism has a genome where the recombinant polynucleotide is stably integrated into the genome and where the recombinant polynucleotide contains a nucleotide sequence encoding an alginate lyase, a DEHU reductase, and an alginate transporter and where integration of the recombinant polynucleotide into the genome modifies the unicellular organism to be able to grow on alginate-containing or alginate-derived media.

One aspect of the present disclosure provides a unicellular organism containing a recombinant polynucleotide where the unicellular organism has a genome where the recombinant polynucleotide is stably integrated into the genome and where the recombinant polynucleotide contains a nucleotide sequence encoding an endo-type cellulase, an exo-type cellulase, a β-glucosidase, and a cellulose/cellobiose transporter and where integration of the one or more heterologous genes into the genome modifies the unicellular organism to be able to grow on cellulose/cellobiose-containing media.

Another aspect of the present disclosure provides a unicellular organism containing a recombinant polynucleotide where the unicellular organism has a genome where the recombinant polynucleotide is stably integrated into the genome, is at least 11 kilobases in size, and has a nucleotide sequence encoding one or more heterologous genes. In certain embodiments, the size of the recombinant polynucleotide is at least 12, 13, 14, 15, 16, 17, 18, 19, 20, 30, 40, 50, 100 kb (including all integers and decimal points in between, e.g., 1.2, 1.3, 1.4, 1.5, 5.5, 5.6, 5.7, 60, 70, etc.) in size. In some embodiments, the size of the recombinant polynucleotide is at least 11 kilobases. In some embodiments, the size of the recombinant polynucleotide is selected from: A) at least 12 kilobases; B) at least 13 kilobases; and C) at least 14 kilobases. In some embodiments, the one or more heterologous genes integrated into the genome is an alginate lyase, a DEHU reductase, and/or an alginate transporter and integration of the one or more heterologous genes into the genome modifies the unicellular organism to be able to grow on alginate-containing or alginate-derived media. In other embodiments, the one or more heterologous genes integrated into the genome is an endo-type cellulase, an exo-type cellulase, a β-glucosidase, and/or a cellulose/cellobiose transporter and integration of the one or more heterologous genes into the genome modifies the unicellular organism to be able to grow on cellulose/cellobiose-containing media.

In certain embodiments which may be combined with any of the preceding aspects providing unicellular organisms in any of their embodiments, the recombinant polynucleotide is positioned between two lox sites in the genome. In certain embodiments which may be combined with any of the preceding aspects providing unicellular organisms in any of their embodiments, the unicellular organism is yeast. In certain embodiments which may be combined with the preceding embodiment where the unicellular organism is yeast, the yeast is a Candida, Hansenula, Kluyveromyces, Pichia, Saccharomyces, Schizosaccharomyces, or Yarrowia strain. In certain embodiments which may be combined with the preceding embodiment where the unicellular organism is yeast, the yeast is a Saccharomyces carlsbergensis, Saccharomyces cerevisiae, Saccharomyces diastaticus, Saccharomyces douglasii, Saccharomyces kluyveri, Saccharomyces norbensis, Saccharomyces monacensis, Saccharomyces bayanus, Saccharomyces pastorianus, Saccharomyces pombe, or Saccharomyces oviformis strain. In other embodiments which may be combined with the preceding embodiment where the unicellular organism is yeast, the yeast is Kluyveromyces lactis, Kluyveromyces fragilis, Kluyveromyces marxiamus, Pichia stipitis, Candida shehatae, or Candida tropicalis. In yet other embodiments which may be combined with the preceding embodiment where the unicellular organism is yeast, the yeast may be Yarrowia lipolytica, Brettanomyces custersii, or Zygosaccharomyces roux. In other embodiments which may be combined with any of the preceding aspects providing unicellular organisms, the unicellular organism is bacteria. In some embodiments which may be combined with the preceding embodiment where the unicellular organism is bacteria, the bacteria may be one of the following: Acetobacter aceti, Achromobacter, Acidiphilium, Acinetobacter, Actinomadura, Actinoplanes, Aeropyrum pernix, Agrobacterium, Alcaligenes, Ananas comosus (M), Arthrobacter, Aspargillus niger, Aspargillus oryze, Aspergillus melleus, Aspergillus pulverulentus, Aspergillus saitoi, Aspergillus sojea, Aspergillus usamii, Bacillus alcalophilus, Bacillus amyloliquefaciens, Bacillus brevis, Bacillus circulans, Bacillus clausii, Bacillus lentus, Bacillus licheniformis, Bacillus macerans, Bacillus stearothermophilus, Bacillus subtilis, Bifidobacterium, Brevibacillus brevis, Burkholderia cepacia, Candida cylindracea, Candida rugosa, Carica papaya (L), Cellulosimicrobium, Cephalosporium, Chaetomium erraticum, Chaetomium gracile, Clostridium, Clostridium butyricum, Clostridium acetobutylicum, Clostridium thermocellum, Corynebacterium (glutamicum), Corynebacterium efficiens, Escherichia coli, Enterococcus, Erwina chrysanthemi, Gliconobacter, Gluconacetobacter, Haloarcula, Humicola insolens, Humicola nsolens, Kitasatospora setae, Klebsiella, Klebsiella oxytoca, Kluyveromyces, Kluyveromyces fragilis, Kluyveromyces lactis, Kocuria, Lactlactis, Lactobacillus, Lactobacillus fermentum, Lactobacillus sake, Lactococcus, Lactococcus lactis, Leuconostoc, Methylocystis, Methanolobus siciliae, Methanogenium organophilum, Methanobacterium bryantii, Microbacterium imperiale, Micrococcus lysodeikticus, Microlunatus, Mucor javanicus, Mycobacterium, Myrothecium, Nitrobacter, Nitrosomonas, Nocardia, Papaya carica, Pediococcus, Pediococcus halophilus, Penicillium, Penicillium camemberti, Penicillium citrinum, Penicillium emersonii, Penicillium roqueforti, Penicillum lilactinum, Penicillum multicolor, Paracoccus pantotrophus, Propionibacterium, Pseudomonas, Pseudomonas fluorescens, Pseudomonas denitrificans, Pyrococcus, Pyrococcus furiosus, Pyrococcus horikoshii, Rhizobium, Rhizomucor miehei, Rhizomucor pusillus Lindt, Rhizopus, Rhizopus delemar, Rhizopus japonicus, Rhizopus niveus, Rhizopus oryzae, Rhizopus oligosporus, Rhodococcus, Saccharomyces cerevisiae, Sclerotina libertina, Sphingobacterium multivorum, Sphingobium, Sphingomonas, Streptococcus, Streptococcus thermophilus Y-1, Streptomyces, Streptomyces griseus, Streptomyces lividans, Streptomyces murinus, Streptomyces rubiginosus, Streptomyces violaceoruber, Streptoverticillium mobaraense, Tetragenococcus, Thermus, Thiosphaera pantotropha, Trametes, Trichoderma, Trichoderma longibrachiatum, Trichoderma reesei, Trichoderma viride, Trichosporon penicillatum, Vibrio alginolyticus, Xanthomonas, Zygosaccharomyces rouxii, Zymomonas, and Zymomonus mobilis. In some embodiments which may be combined with the preceding embodiment where the unicellular organism is bacteria, the bacteria are gram-negative. In some embodiments which may be combined with the preceding embodiment where the unicellular organism is bacteria, the bacteria are classified in the family of Enterobacteriaceae. In some embodiments which may be combined with the preceding embodiment where the bacteria is bacteria classified in the family of Enterobacteriaceae, the bacteria are Aranicola, Arsenophonus, Averyella, Biostraticola, Brenneria, Buchnera, Budvicia, Buttiauxella, Candidatus, Curculioniphilus, Cuticobacterium, Candidatus Ishikawaella, Macropleicola, Phlomobacter, Candidatus Riesia, Candidatus Stammerula, Cedecea, Citrobacter, Cronobacter, Dickeya, Edwardsiella, Enterobacter, Erwinia, Escherichia, Ewingella, Grimontella, Hafnia, Klebsiella, Kluyvera, Leclercia, Leminorella, Margalefia, Moellerella, Morganella, Obesumbacterium, Pantoea, Pectobacterium, Photorhabdus, Phytobacter, Plesiomonas, Pragia, Proteus, Providencia, Rahnella, Raoultella, Salmonella, Samsonia, Serratia, Shigella, Sodalis, Tatumella, Thorasellia, Tiedjeia, Trabulsiella, Wigglesworthia, Xenorhabdus, Yersinia, or Yokenella.

One aspect of the present disclosure provides a gram-negative bacterial strain containing a recombinant polynucleotide where the gram-negative bacterial strain has a genome where the recombinant polynucleotide is stably integrated into the genome, is positioned between two lox sites in the genome, and has a nucleotide sequence encoding one or more heterologous genes. In certain embodiments, the size of the polynucleotide is at least 1, 2, 3, 4, 5, 6, 7, 8, 9, 10, 11, 12, 13, 14, 15, 16, 17, 18, 19, 20, 30, 40, 50, 100 kb (including all integers and decimal points in between, e.g., 1.2, 1.3, 1.4, 1.5, 5.5, 5.6, 5.7, 60, 70, etc.). In some embodiments, the size of the recombinant polynucleotide is at least 11 kilobases. In certain embodiments, the one or more heterologous genes integrated into the genome is an alginate lyase, a DEHU reductase, and/or an alginate transporter and integration of the one or more heterologous genes into the genome modifies the gram-negative bacterial strain to be able to grow on alginate-containing or alginate-derived media. In other embodiments, the one or more heterologous genes integrated into the genome is an endo-type cellulase, an exo-type cellulase, a β-glucosidase, and/or a cellulose/cellobiose transporter and integration of the one or more heterologous genes into the genome modifies the gram-negative bacterial strain to be able to grow on cellulose/cellobiose-containing media. In certain embodiments, the gram-negative bacterial strain is an E. coli strain.

One aspect of the present disclosure provides a method of integrating a recombinant polynucleotide of at least 11 kilobases in the genome of a unicellular organism by: A) providing a unicellular organism containing a genome having a first lox site and a second lox site integrated in the genome of the unicellular organism, where the first lox site has a different sequence from the second lox site such that the first and second lox sites are incapable of recombining with each other; B) transforming the unicellular organism, with a first plasmid and a second plasmid, where the first plasmid contains a recombinant polynucleotide having a nucleotide sequence encoding one or more heterologous genes, where the size of the recombinant polynucleotide is at least 11 kilobases and where the recombinant polynucleotide is bounded by a third lox site and a fourth lox site where the third lox site has the same sequence as the first lox site and the fourth lox site has the same sequence as the second lox site, and where the second plasmid encodes Cre recombinase; C) culturing the unicellular organism under conditions such that Cre recombinase is expressed, where Cre recombinase expression results in homologous recombination between the first and third lox sites and between the second and fourth lox sites and integration of the recombinant polynucleotide into the genome of the unicellular organism in between the first and second lox sites. In certain embodiments, the method further includes D) growing the unicellular organism in media and under conditions where the one or more heterologous genes are expressed and a commodity chemical is produced; and E) collecting the commodity chemical.

Another aspect of the present disclosure provides a method of integrating a recombinant polynucleotide of at least 11 kilobases in the genome of a unicellular organism by: A) providing a unicellular organism containing a genome having a first lox site and a second lox site integrated in the genome of the unicellular organism, where the first lox site has a different sequence from the second lox site such that the first and second lox sites are incapable of recombining with each other, and containing a plasmid encoding Cre recombinase; B) providing a donor cell containing a recombinant polynucleotide, the recombinant polynucleotide having a nucleotide sequence encoding one or more heterologous genes, where the size of the recombinant polynucleotide is at least 11 kilobases and where the recombinant polynucleotide is bounded by a third lox site and a fourth lox site where the third lox site has the same sequence as the first lox site and the fourth lox site has the same sequence as the second lox site; C) infecting the donor cell with a phage such that phage particles containing the recombinant polynucleotide are produced and released from the donor cell; D) culturing the unicellular organism such that Cre recombinase is expressed; E) infecting the unicellular organism expressing Cre recombinase with the phage particles, where Cre recombinase expression results in homologous recombination between the first and third lox sites and between the second and fourth lox sites and integration of the recombinant polynucleotide into the genome of the unicellular organism in between the first and second lox sites. In certain embodiments, the method further includes F) growing the unicellular organism in media and under conditions where the one or more heterologous genes are expressed and a commodity chemical is produced; and G) collecting the commodity chemical.

Another aspect of the present disclosure provides a method of producing a commodity chemical by: A) providing a unicellular organism containing a genome having a first lox site and a second lox site integrated in the genome of the unicellular organism, where the first lox site has a different sequence from the second lox site such that the first and second lox sites are incapable of recombining with each other; B) transforming the unicellular organism with a first plasmid and a second plasmid, where the first plasmid has a recombinant polynucleotide containing a nucleotide sequence encoding one or more heterologous genes, where the size of the recombinant polynucleotide is at least 11 kilobases and where the recombinant polynucleotide is bounded by a third lox site and a fourth lox site where the third lox site has the same sequence as the first lox site and the fourth lox site has the same sequence as the second lox site, and where the second plasmid encodes Cre recombinase; C) culturing the unicellular organism under conditions such that Cre recombinase is expressed, where Cre recombinase expression results in homologous recombination between the first and third lox sites and between the second and fourth lox sites and integration of the recombinant polynucleotide into the genome of the unicellular organism in between the first and second lox sites; D) growing the unicellular organism in media and under conditions where the one or more heterologous genes is expressed and a commodity chemical is produced; and E) collecting the commodity chemical.

Yet another aspect of the present disclosure provides a method of producing a commodity chemical by: A) providing a unicellular organism containing a genome having a first lox site and a second lox site integrated in the genome of the unicellular organism, where the first lox site has a different sequence from the second lox site such that the first and second lox sites are incapable of recombining with each other, and containing a plasmid encoding Cre recombinase; B) providing a donor cell containing a recombinant polynucleotide, the recombinant polynucleotide having a nucleotide sequence encoding one or more heterologous genes, where the size of the recombinant polynucleotide is at least 11 kilobases and where the recombinant polynucleotide is bounded by a third lox site and a fourth lox site where the third lox site has the same sequence as the first lox site and the fourth lox site has the same sequence as the second lox site; C) infecting the donor cell with a phage such that phage particles containing the recombinant polynucleotide are produced and released from the donor cell; D) culturing the unicellular organism such that Cre recombinase is expressed; E) infecting the unicellular organism expressing Cre recombinase with the phage particles, where Cre recombinase expression results in homologous recombination between the first and third lox sites and between the second and fourth lox sites and integration of the recombinant polynucleotide into the genome of the unicellular organism in between the first and second lox sites; F) growing the unicellular organism in media and under conditions where the one or more heterologous genes is expressed and a commodity chemical is produced; and G) collecting the commodity chemical.

Yet another aspect of the present disclosure provides a method of integrating a recombinant polynucleotide in the genome of gram-negative bacteria by: A) providing gram-negative bacteria containing a genome having a first lox site and a second lox site integrated in the genome of the bacteria, where the first lox site has a different sequence from the second lox site such that the first and second lox sites are incapable of recombining with each other; B) transforming the bacteria, with a first plasmid and a second plasmid, where the first plasmid has a recombinant polynucleotide containing a nucleotide sequence encoding one or more heterologous genes, where the recombinant polynucleotide is bounded by a third lox site and a fourth lox site where the third lox site has the same sequence as the first lox site and the fourth lox site has the same sequence as the second lox site, and where the second plasmid encodes Cre recombinase; C) culturing the bacteria under conditions such that Cre recombinase is expressed, where Cre recombinase expression results in homologous recombination between the first and third lox sites and between the second and fourth lox sites and integration of the recombinant polynucleotide into the genome of the bacteria in between the first and second lox sites. In certain embodiments, the method further includes D) growing the bacteria in media and under conditions where the one or more heterologous genes are expressed and a commodity chemical is produced; and E) collecting the commodity chemical.

One aspect of the present disclosure provides a method of integrating a recombinant polynucleotide in the genome of gram-negative bacteria by: A) providing gram-negative bacteria containing a genome having a first lox site and a second lox site integrated in the genome of the bacteria, where the first lox site has a different sequence from the second lox site such that the first and second lox sites are incapable of recombining with each other, and containing a plasmid encoding Cre recombinase; B) providing a donor cell containing recombinant polynucleotide, the recombinant polynucleotide having a nucleotide sequence encoding one or more heterologous genes, where the recombinant polynucleotide is bounded by a third lox site and a fourth lox site where the third lox site has the same sequence as the first lox site and the fourth lox site has the same sequence as the second lox site; C) infecting the donor cell with a phage such that phage particles containing the recombinant polynucleotide are produced and released from the donor cell; D) culturing the bacteria such that Cre recombinase is expressed; E) infecting the bacteria expressing Cre recombinase with the phage particles, where Cre recombinase expression results in homologous recombination between the first and third lox sites and between the second and fourth lox sites and integration of the recombinant polynucleotide into the genome of the bacteria in between the first and second lox sites. In certain embodiments, the method further includes F) growing the bacteria in media and under conditions where the one or more heterologous genes are expressed and a commodity chemical is produced; and G) collecting the commodity chemical.

Another aspect of the present disclosure provides a method of producing a commodity chemical by: A) providing gram-negative bacteria containing a genome having a first lox site and a second lox site integrated in the genome of the bacteria, where the first lox site has a different sequence from the second lox site such that the first and second lox sites are incapable of recombining with each other; B) transforming the bacteria, with a first plasmid and a second plasmid, where the first plasmid has a recombinant polynucleotide containing a nucleotide sequence encoding one or more heterologous genes, where the recombinant polynucleotide is bounded by a third lox site and a fourth lox site where the third lox site has the same sequence as the first lox site and the fourth lox site has the same sequence as the second lox site, and where the second plasmid encodes Cre recombinase; C) culturing the bacteria under conditions such that Cre recombinase is expressed, where Cre recombinase expression results in homologous recombination between the first and third lox sites and between the second and fourth lox sites and integration of the recombinant polynucleotide into the genome of the bacteria in between the first and second lox sites; D) growing the bacteria in media and under conditions where the one or more heterologous genes is expressed and a commodity chemical is produced; and E) collecting the commodity chemical.

Yet another aspect of the present disclosure provides a method of producing a commodity chemical by: A) providing gram-negative bacteria containing a genome having a first lox site and a second lox site integrated in the genome of the bacteria, where the first lox site has a different sequence from the second lox site such that the first and second lox sites are incapable of recombining with each other, and containing a plasmid encoding Cre recombinase; B) providing a donor cell containing recombinant polynucleotide, the recombinant polynucleotide having a nucleotide sequence encoding one or more heterologous genes, where the recombinant polynucleotide is bounded by a third lox site and a fourth lox site where the third lox site has the same sequence as the first lox site and the fourth lox site has the same sequence as the second lox site; C) infecting the donor cell with a phage such that phage particles containing the recombinant polynucleotide are produced and released from the donor cell; D) culturing the bacteria such that Cre recombinase is expressed; E) infecting the bacteria expressing Cre recombinase with the phage particles, where Cre recombinase expression results in homologous recombination between the first and third lox sites and between the second and fourth lox sites and integration of the recombinant polynucleotide into the genome of the bacteria in between the first and second lox sites; F) growing the bacteria in media and under conditions where the one or more heterologous genes is expressed and a commodity chemical is produced; and G) collecting the commodity chemical.

In embodiments which may be combined with any of the preceding aspects in any of their embodiments providing a method where a phage is used, the phage is P1vir.

In embodiments which may be combined with any of the preceding aspects in any of their embodiments providing a method where gram-negative bacteria are used, the gram-negative bacteria are E. coli. In embodiments which may be combined with any of the preceding aspects in any of their embodiments providing a method where gram-negative bacteria are used, the size of the recombinant polynucleotide is at least 11 kilobases.

In embodiments which may be combined with any of the preceding aspects in any of their embodiments providing a method where a unicellular organism is used, the size of the recombinant polynucleotide is selected from: A) at least 12 kilobases; B) at least 13 kilobases; and C) at least 14 kilobases. In embodiments which may be combined with any of the preceding aspects in any of their embodiments providing a method where a unicellular organism is used, the unicellular organism is yeast. In embodiments which may be combined with the preceding embodiment where the unicellular organism is yeast, the yeast is a Candida, Hansenula, Kluyveromyces, Pichia, Saccharomyces, Schizosaccharomyces, or Yarrowia strain. In other embodiments which may be combined with the preceding embodiment where the unicellular organism is yeast, the yeast is a Saccharomyces carlsbergensis, Saccharomyces cerevisiae, Saccharomyces diastaticus, Saccharomyces douglasii, Saccharomyces kluyveri, Saccharomyces norbensis, Saccharomyces monacensis, Saccharomyces bayanus, Saccharomyces pastorianus, Saccharomyces pombe, or Saccharomyces oviformis strain. In certain embodiments which may be combined with the preceding embodiment where the unicellular organism is yeast, the yeast is Kluyveromyces lactis, Kluyveromyces fragilis, Kluyveromyces marxiamus, Pichia stipitis, Candida shehatae, or Candida tropicalis. In other embodiments which may be combined with the preceding embodiment where the unicellular organism is yeast, the yeast may be Yarrowia lipolytica, Brettanomyces custersii, or Zygosaccharomyces roux. In embodiments which may be combined with any of the preceding aspects in any of their embodiments providing a method where a unicellular organism is used, the unicellular organism is bacteria. In certain embodiments which may be combined with the preceding embodiment where the unicellular organism is bacteria, the bacteria is one of the following: Acetobacter aceti, Achromobacter, Acidiphilium, Acinetobacter, Actinomadura, Actinoplanes, Aeropyrum pernix, Agrobacterium, Alcaligenes, Ananas comosus (M), Arthrobacter, Aspargillus niger, Aspargillus oryze, Aspergillus melleus, Aspergillus pulverulentus, Aspergillus saitoi, Aspergillus sojea, Aspergillus usamii, Bacillus alcalophilus, Bacillus amyloliquefaciens, Bacillus brevis, Bacillus circulans, Bacillus clausii, Bacillus lentus, Bacillus licheniformis, Bacillus macerans, Bacillus stearothermophilus, Bacillus subtilis, Bifidobacterium, Brevibacillus brevis, Burkholderia cepacia, Candida cylindracea, Candida rugosa, Carica papaya (L), Cellulosimicrobium, Cephalosporium, Chaetomium erraticum, Chaetomium gracile, Clostridium, Clostridium butyricum, Clostridium acetobutylicum, Clostridium thermocellum, Corynebacterium (glutamicum), Corynebacterium efficiens, Escherichia coli, Enterococcus, Erwina chrysanthemi, Gliconobacter, Gluconacetobacter, Haloarcula, Humicola insolens, Humicola nsolens, Kitasatospora setae, Klebsiella, Klebsiella oxytoca, Kluyveromyces, Kluyveromyces fragilis, Kluyveromyces lactis, Kocuria, Lactlactis, Lactobacillus, Lactobacillus fermentum, Lactobacillus sake, Lactococcus, Lactococcus lactis, Leuconostoc, Methylocystis, Methanolobus siciliae, Methanogenium organophilum, Methanobacterium bryantii, Microbacterium imperiale, Micrococcus lysodeikticus, Microlunatus, Mucor javanicus, Mycobacterium, Myrothecium, Nitrobacter, Nitrosomonas, Nocardia, Papaya carica, Pediococcus, Pediococcus halophilus, Penicillium, Penicillium camemberti, Penicillium citrinum, Penicillium emersonii, Penicillium roqueforti, Penicillum lilactinum, Penicillum multicolor, Paracoccus pantotrophus, Propionibacterium, Pseudomonas, Pseudomonas fluorescens, Pseudomonas denitrificans, Pyrococcus, Pyrococcus furiosus, Pyrococcus horikoshii, Rhizobium, Rhizomucor miehei, Rhizomucor pusillus Lindt, Rhizopus, Rhizopus delemar, Rhizopus japonicus, Rhizopus niveus, Rhizopus oryzae, Rhizopus oligosporus, Rhodococcus, Saccharomyces cerevisiae, Sclerotina libertina, Sphingobacterium multivorum, Sphingobium, Sphingomonas, Streptococcus, Streptococcus thermophilus Y-1, Streptomyces, Streptomyces griseus, Streptomyces lividans, Streptomyces murinus, Streptomyces rubiginosus, Streptomyces violaceoruber, Streptoverticillium mobaraense, Tetragenococcus, Thermus, Thiosphaera pantotropha, Trametes, Trichoderma, Trichoderma longibrachiatum, Trichoderma reesei, Trichoderma viride, Trichosporon penicillatum, Vibrio alginolyticus, Xanthomonas, Zygosaccharomyces rouxii, Zymomonas, and Zymomonus mobilis. In certain embodiments which may be combined with the preceding embodiment where the unicellular organism is bacteria, the bacteria are gram-negative. In some embodiments which may be combined with the preceding embodiment where the unicellular organism is bacteria, the bacteria are classified in the family of Enterobacteriaceae. In certain embodiments which may be combined with the preceding embodiment where the bacteria are classified in the family of Enterobacteriaceae, the bacteria are Aranicola, Arsenophonus, Averyella, Biostraticola, Brenneria, Buchnera, Budvicia, Buttiauxella, Candidatus, Curculioniphilus, Cuticobacterium, Candidatus Ishikawaella, Macropleicola, Phlomobacter, Candidatus Riesia, Candidatus Stammerula, Cedecea, Citrobacter, Cronobacter, Dickeya, Edwardsiella, Enterobacter, Erwinia, Escherichia, Ewingella, Grimontella, Hafnia, Klebsiella, Kluyvera, Leclercia, Leminorella, Margalefia, Moellerella, Morganella, Obesumbacterium, Pantoea, Pectobacterium, Photorhabdus, Phytobacter, Plesiomonas, Pragia, Proteus, Providencia, Rahnella, Raoultella, Salmonella, Samsonia, Serratia, Shigella, Sodalis, Tatumella, Thorasellia, Tiedjeia, Trabulsiella, Wigglesworthia, Xenorhabdus, Yersinia, or Yokenella. In some embodiments which may be combined with the preceding embodiment where the unicellular organism is bacteria, the bacteria are Escherichia coli (E. coli).

In some embodiments which may be combined with any of the preceding aspects providing methods of integrating a recombinant polynucleotide in any of their embodiments, the one or more heterologous genes integrated into the genome is an alginate lyase, a DEHU reductase, and/or an alginate transporter and integration of the one or more heterologous genes into the genome modifies the unicellular organism or gram-negative bacterial strain to be able to grow on alginate-containing or alginate-derived media. In other embodiments which may be combined with any of the preceding aspects providing methods of integrating a recombinant polynucleotide in any of their embodiments, the one or more heterologous genes integrated into the genome are an alginate lyase, a DEHU reductase, and an alginate transporter and integration of the heterologous genes into the genome modifies the unicellular organism or gram-negative bacterial strain to be able to grow on alginate-containing or alginate-derived media. In other embodiments which may be combined with any of the preceding aspects providing methods of integrating a recombinant polynucleotide in any of their embodiments, the one or more heterologous genes integrated into the genome is an endo-type cellulase, an exo-type cellulase, a β-glucosidase, and/or a cellulose/cellobiose transporter and integration of the one or more heterologous genes into the genome modifies the unicellular organism or gram-negative bacterial strain to be able to grow on cellulose/cellobiose-containing media.

In some embodiments which may be combined with any of the preceding aspects providing methods of producing a commodity chemical in any of their embodiments, the one or more heterologous genes integrated into the genome is an alginate lyase, a DEHU reductase, and/or an alginate transporter and the media contains, or is derived from, alginate. In preferred embodiments which may be combined with any of the preceding aspects providing methods of producing a commodity chemical in any of their embodiments, the one or more heterologous genes integrated into the genome are an alginate lyase, a DEHU reductase, and an alginate transporter and the media contains, or is derived from, alginate. In other embodiments which may be combined with any of the preceding aspects providing methods of producing a commodity chemical in any of their embodiments, the one or more heterologous genes integrated into the genome is an endo-type cellulase, an exo-type cellulase, a β-glucosidase, and/or a cellulose/cellobiose transporter and the media contains cellulose/cellobiose. In some embodiments which may be combined with any of the preceding aspects providing methods of producing a commodity chemical in any of their embodiments, the commodity chemical is ethanol. In some embodiments which may be combined with any of the preceding aspects providing methods of producing a commodity chemical in any of their embodiments, the commodity chemical is isobutanol. In other embodiments which may be combined with any of the preceding aspects providing methods of producing a commodity chemical in any of their embodiments, the commodity chemical is n-butanol. In yet other embodiments which may be combined with any of the preceding aspects providing methods of producing a commodity chemical in any of their embodiments, the commodity chemical is 2-butanol.

One aspect of the present disclosure provides a method of integrating a recombinant polynucleotide in the genome of an E. coli strain by: A) providing an E. coli strain containing a genome having a first lox site and a second lox site integrated in the genome of the E. coli strain, where the first lox site has a different sequence from the second lox site such that the first and second lox sites are incapable of recombining with each other; B) transforming the E. coli strain, with a first plasmid and a second plasmid, where the first plasmid has a recombinant polynucleotide containing a nucleotide sequence encoding one or more heterologous genes, where the recombinant polynucleotide is bounded by a third lox site and a fourth lox site where the third lox site has the same sequence as the first lox site and the fourth lox site has the same sequence as the second lox site, and where the second plasmid encodes Cre recombinase; C) culturing the bacteria under conditions such that Cre recombinase is expressed, where Cre recombinase expression results in homologous recombination between the first and third lox sites and between the second and fourth lox sites and integration of the recombinant polynucleotide into the genome of the E. coli strain in between the first and second lox sites. In certain embodiments, the method further includes D) growing the E. coli strain in media and under conditions where the one or more heterologous genes are expressed and a commodity chemical is produced; and E) collecting the commodity chemical.

Another aspect of the present disclosure provides a method of integrating a recombinant polynucleotide in the genome of an E. coli strain by: A) providing an E. coli strain containing a genome having a first lox site and a second lox site integrated in the genome of the E. coli strain, where the first lox site has a different sequence from the second lox site such that the first and second lox sites are incapable of recombining with each other, and containing a plasmid encoding Cre recombinase; B) providing a donor cell containing recombinant polynucleotide, the recombinant polynucleotide having a nucleotide sequence encoding one or more heterologous genes, where the recombinant polynucleotide is bounded by a third lox site and a fourth lox site where the third lox site has the same sequence as the first lox site and the fourth lox site has the same sequence as the second lox site; C) infecting the donor cell with a phage such that phage particles containing the recombinant polynucleotide are produced and released from the donor cell; D) culturing the E. coli strain such that Cre recombinase is expressed; E) infecting the E. coli strain expressing Cre recombinase with the phage particles, where Cre recombinase expression results in homologous recombination between the first and third lox sites and between the second and fourth lox sites and integration of the recombinant polynucleotide into the genome of the E. coli strain in between the first and second lox sites. In certain embodiments, the method further includes F) growing the E. coli strain in media and under conditions where the one or more heterologous genes are expressed and a commodity chemical is produced; and G) collecting the commodity chemical.

In some embodiments which may be combined with any of the preceding embodiments, the one or more heterologous genes integrated into the genome are an alginate lyase, a DEHU reductase, and/or an alginate transporter and where integration of the one or more heterologous genes into the genome modifies the E. coli strain to be able to grow on alginate-containing or alginate-derived media. In other embodiments which may be combined with any of the preceding embodiments, the one or more heterologous genes integrated into the genome are an alginate lyase, a DEHU reductase, and an alginate transporter and where integration of the heterologous genes into the genome modifies the E. coli strain to be able to grow on alginate-containing or alginate-derived media. In other embodiments which may be combined with any of the preceding embodiments, the one or more heterologous genes integrated into the genome are an endo-type cellulase, an exo-type cellulase, a β-glucosidase, and/or a cellulose/cellobiose transporter and where integration of the one or more heterologous genes into the genome modifies the E. coli strain to be able to grow on cellulose/cellobiose-containing media. In other embodiments which may be combined with any of the preceding embodiments, the phage is P1vir. In other embodiments which may be combined with any of the preceding embodiments, the size of the recombinant polynucleotide is at least 11 kilobases. In other embodiments which may be combined with any of the preceding embodiments, the commodity chemical is ethanol.

### BRIEF DESCRIPTION OF THE DRAWINGS

FIG. 1 shows Cre-lox recombination of large genetic fragments into bacterial genomes. Step 1 shows the incorporation of the cat gene flanked by two lox sites (loxP and lox5171) into the bacterial genome through arabinose induction of the λ-RED recombination genes (on pKD46). Step 2 shows the plasmid-based method in which this strain was subsequently transformed with both pJW168 and pALG2.3.4 plasmids and then grown at 30° C. with IPTG to induce Cre recombinase expression. Step 3 shows the phage-based method in which P1vir lysates were prepared from a donor strain containing the (donor) plasmid with the lox-flanked genetic fragment and the lysates subsequently used to infect a recipient strain induced by IPTG for Cre recombinase expression. In both Steps 1 and 2, the temperature-sensitive plasmid pJW168 was lost following plating on kanamycin and growth at 37° C.

FIG. 2 shows the verification and evaluation of integrated constructs. Part (a) shows the colony PCR verification of integrated strains across the ldhA junction. BAL1075 ldhA::loxP-Cm-lox5171 was used as a negative control for the PCR reactions. Expected product sizes were as follows—A (left end verification): 588 bp; B (right end verification): 608 bp; C (ldhA::loxP-Cm-lox5171 cassette): no product expected if correct or 471 bp if integration failed. Similar results were found for all ten strains tested; only one representative set of data is shown. Part (b) shows the growth of five integration clones on 2% degraded alginate medium. BAL1075 was used as a negative control for these growth assays. Clones 1-3 were derived from the plasmid-based method for integration whereas clones 4-5 were obtained through the phage-based method. Part (c) shows the relationship between integration efficiency and cassette length. Efficiency was represented as the percent colonies that were found to be sensitive to chloramphenicol. Square symbols indicate clones obtained using plasmid-based recombination and diamond symbols indicate clones obtained using phage-based recombination.

FIG. 3 shows the effect of chromosomal location and copy number on growth on alginate. Single copy and double copy integration strains were grown on 2% degraded alginate medium. Percent distances from the chromosomal origin (oriC) for each integration locus were as follows: ldhA—92.5%; int(gidB-atpI)—0.1%, int(mraZ-fruR)—33.3%.

FIG. 4 shows a schematic of the alginate metabolic pathway.

### DETAILED DESCRIPTION

The present disclosure relates to methods of integrating recombinant polynucleotides into the genomes of unicellular organisms by using the Cre-lox recombination system in order to express heterologous genes. The present disclosure further relates to methods of producing commodity chemicals, e.g., ethanol, by the use of such modified organisms. The present disclosure also relates to unicellular organisms that have recombinant polynucleotides containing heterologous genes integrated into their genomes.

### I. Methods Using Cre-lox Recombination System

The present disclosure provides a method of integrating a recombinant polynucleotide in the genome of a unicellular organism by: A) providing a unicellular organism having a genome containing a first lox site and a second lox site integrated in the genome of the unicellular organism where the first lox site has a different sequence from the second lox site such that the first and second lox sites are incapable of recombining with each other; B) transforming the unicellular organism with a first plasmid and a second plasmid where the first plasmid comprises a recombinant polynucleotide containing a nucleotide sequence encoding one or more heterologous genes bounded by a third lox site and a fourth lox site where the third lox site has the same sequence as the first lox site and the fourth lox site has the same sequence as the second lox site and where the second plasmid encodes Cre recombinase; C) culturing the unicellular organism under conditions such that Cre recombinase is expressed where Cre recombinase expression results in homologous recombination between the first and third lox sites and between the second and fourth lox sites and integration of the recombinant polynucleotide into the genome of the unicellular organism in between the first and second lox sites.

The present disclosure also provides a method of integrating a recombinant polynucleotide in the genome of a unicellular organism by: A) providing a unicellular organism having a genome containing a first lox site and a second lox site integrated in the genome of the unicellular organism where the first lox site contains a different sequence from the second lox site such that the first and second lox sites are incapable of recombining with each other, and having a plasmid encoding Cre recombinase; B) providing a donor cell containing a recombinant polynucleotide having a nucleotide sequence encoding one or more heterologous genes bounded by a third lox site and a fourth lox site where the third lox site has the same sequence as the first lox site and the fourth lox site has the same sequence as the second lox site; C) infecting the donor cell with a phage such that phage particles containing the recombinant polynucleotide are produced and released from the donor cell; D) culturing the unicellular organism such that Cre recombinase is expressed; E) infecting the unicellular organism expressing Cre recombinase with the phage particles where Cre recombinase expression results in homologous recombination between the first and third lox sites and between the second and fourth lox sites and integration of the recombinant polynucleotide into the genome of the unicellular organism in between the first and second lox sites.

In some embodiments, the unicellular organism is gram-negative bacteria, such as E. coli, or yeast.

A. Cre-lox Recombination System

As used herein the expression “lox site” means a nucleotide sequence at which the Cre recombinase can catalyze a site-specific recombination. Examples of the lox site include the canonical lox sequence loxP, lox5171, etc. Various mutated sequences of lox sites can also be used so long as such sequences remain recognizable by the Cre recombinase (Lee & Saito, 1998).

In certain embodiments of the present disclosure, the gene encoding Cre recombinase is provided in trans under the control of either constitutive, inducible or developmentally-regulated promoters (see, e.g., Baubonis & Sauer, Nucleic Acids Res., 21, 2025-2029 (1993); Dang et al., Develop. Genet., 13, 367-375 (1992); Chou et al., Genetics, 131, 643-653 (1992); Morris et al., Nucleic Acids Res., 19, 5895-5900 (1991)). Preferably the Cre coding sequence has the coding sequence of bacteriophage P1 recombinase Cre, or various mutations of this sequence such as described in the art (e.g., Wierzbicki et al., J. Mol. Biol., 195, 785-794 (1987); Abremski et al., J. Mol. Biol., 202, 59-66 (1988); Abremski et al., J. Mol. Biol., 184, 211-20 (1988); Abremski et al., Protein Engineering, 5, 87-91 (1992) Hoess et al., Proc. Natl. Acad. Sci., 84, 6840-6844 (1987); Sternberg et al., J. Mol. Biol., 187, 197-212 (1986)). Further mutations of this Cre coding sequence may be employed so long as variant proteins resulting from such mutations are capable of effecting recombination at lox sites.

Transforming a cell refers generally to the permanent, heritable alteration in a cell resulting from the uptake and incorporation of foreign DNA, e.g., in the form of a plasmid, into the genome of unicellular organisms. In the present disclosure, the recombinant polynucleotides are introduced into a unicellular organism, by a number of transformation methods including, but not limited to, electroporation, lipid-assisted transformation or transfection (“lipofection”), chemically mediated transfection (e.g., using calcium chloride and/or calcium phosphate), lithium acetate-mediated transformation (e.g., of host-cell protoplasts), biolistic “gene gun” transformation, PEG-mediated transformation (e.g., of host-cell protoplasts), protoplast fusion (e.g., using bacterial or eukaryotic protoplasts), liposome-mediated transformation, Agrobacterium tumefaciens, adenovirus or other viral or phage transformation or transduction.

In the present disclosure, the plasmid may be any plasmid compatible with the unicellular organism. The plasmid may include a reporter gene, such as a green fluorescent protein (GFP), which can be either fused in frame to one or more of the encoded polypeptides, or expressed separately. The plasmid can also include a selection marker such as an antibiotic resistance gene that can be used for selection of suitable transformants.

In certain embodiments, the phage is bacteriophage P1 or its derivatives. In preferred embodiments, the phage is P1vir. Other phages specific to the type of unicellular organism may also be used in the present disclosure.

In the present disclosure, all organisms classified in the family of Enterobacteriaceae can be used as donor cells when phage is used. Examples of genera in the family include Aranicola, Arsenophonus, Averyella, Biostraticola, Brenneria, Buchnera, Budvicia, Buttiauxella, Candidatus, Curculioniphilus, Cuticobacterium, Candidatus Ishikawaella, Macropleicola, Phlomobacter, Candidatus Riesia, Candidatus Stammerula, Cedecea, Citrobacter, Cronobacter, Dickeya, Edwardsiella, Enterobacter, Erwinia, Escherichia, Ewingella, Grimontella, Hafnia, Klebsiella, Kluyvera, Leclercia, Leminorella, Margalefia, Moellerella, Morganella, Obesumbacterium, Pantoea, Pectobacterium, Photorhabdus, Phytobacter, Plesiomonas, Pragia, Proteus, Providencia, Rahnella, Raoultella, Salmonella, Samsonia, Serratia, Shigella, Sodalis, Tatumella, Thorasellia, Tiedjeia, Trabulsiella, Wigglesworthia, Xenorhabdus, Yersinia, and Yokenella.

B. Unicellular Organisms

In certain embodiments of the methods of integrating recombinant polynucleotides in genomes of unicellular organisms described above, the unicellular organism can be yeast or bacteria.

In some embodiments, the yeast is a Candida, Hansenula, Kluyveromyces, Pichia, Saccharomyces, Schizosaccharomyces, or Yarrowia strain. In certain embodiments, the yeast is a Saccharomyces carlsbergensis, Saccharomyces cerevisiae, Saccharomyces diastaticus, Saccharomyces douglasii, Saccharomyces kluyveri, Saccharomyces norbensis, Saccharomyces monacensis, Saccharomyces bayanus, Saccharomyces pastorianus, Saccharomyces pombe, or Saccharomyces oviformis strain. In other preferred embodiments, the yeast is Kluyveromyces lactis, Kluyveromyces fragilis, Kluyveromyces marxiamus, Pichia stipitis, Candida shehatae, or Candida tropicalis. In other embodiments, the yeast may be Yarrowia lipolytica, Brettanomyces custersii, or Zygosaccharomyces roux.

In embodiments where the unicellular organism is bacteria, the bacteria may be one of the following: Acetobacter aceti, Achromobacter, Acidiphilium, Acinetobacter, Actinomadura, Actinoplanes, Aeropyrum pernix, Agrobacterium, Alcaligenes, Ananas comosus (M), Arthrobacter, Aspargillus niger, Aspargillus oryze, Aspergillus melleus, Aspergillus pulverulentus, Aspergillus saitoi, Aspergillus sojea, Aspergillus usamii, Bacillus alcalophilus, Bacillus amyloliquefaciens, Bacillus brevis, Bacillus circulans, Bacillus clausii, Bacillus lentus, Bacillus licheniformis, Bacillus macerans, Bacillus stearothermophilus, Bacillus subtilis, Bifidobacterium, Brevibacillus brevis, Burkholderia cepacia, Candida cylindracea, Candida rugosa, Carica papaya (L), Cellulosimicrobium, Cephalosporium, Chaetomium erraticum, Chaetomium gracile, Clostridium, Clostridium butyricum, Clostridium acetobutylicum, Clostridium thermocellum, Corynebacterium (glutamicum), Corynebacterium efficiens, Escherichia coli, Enterococcus, Erwina chrysanthemi, Gliconobacter, Gluconacetobacter, Haloarcula, Humicola insolens, Humicola nsolens, Kitasatospora setae, Klebsiella, Klebsiella oxytoca, Kluyveromyces, Kluyveromyces fragilis, Kluyveromyces lactis, Kocuria, Lactlactis, Lactobacillus, Lactobacillus fermentum, Lactobacillus sake, Lactococcus, Lactococcus lactis, Leuconostoc, Methylocystis, Methanolobus siciliae, Methanogenium organophilum, Methanobacterium bryantii, Microbacterium imperiale, Micrococcus lysodeikticus, Microlunatus, Mucor javanicus, Mycobacterium, Myrothecium, Nitrobacter, Nitrosomonas, Nocardia, Papaya carica, Pediococcus, Pediococcus halophilus, Penicillium, Penicillium camemberti, Penicillium citrinum, Penicillium emersonii, Penicillium roqueforti, Penicillum lilactinum, Penicillum multicolor, Paracoccus pantotrophus, Propionibacterium, Pseudomonas, Pseudomonas fluorescens, Pseudomonas denitrificans, Pyrococcus, Pyrococcus furiosus, Pyrococcus horikoshii, Rhizobium, Rhizomucor miehei, Rhizomucor pusillus Lindt, Rhizopus, Rhizopus delemar, Rhizopus japonicus, Rhizopus niveus, Rhizopus oryzae, Rhizopus oligosporus, Rhodococcus, Saccharomyces cerevisiae, Sclerotina libertina, Sphingobacterium multivorum, Sphingobium, Sphingomonas, Streptococcus, Streptococcus thermophilus Y-1, Streptomyces, Streptomyces griseus, Streptomyces lividans, Streptomyces murinus, Streptomyces rubiginosus, Streptomyces violaceoruber, Streptoverticillium mobaraense, Tetragenococcus, Thermus, Thiosphaera pantotropha, Trametes, Trichoderma, Trichoderma longibrachiatum, Trichoderma reesei, Trichoderma viride, Trichosporon penicillatum, Vibrio alginolyticus, Xanthomonas, Zygosaccharomyces rouxii, Zymomonas, and Zymomonas mobilis. In some embodiments, the bacteria are gram-negative. In some embodiments, the bacteria are classified in the family of Enterobacteriaceae. Examples of genera in the family include Aranicola, Arsenophonus, Averyella, Biostraticola, Brenneria, Buchnera, Budvicia, Buttiauxella, Candidatus, Curculioniphilus, Cuticobacterium, Candidatus Ishikawaella, Macropleicola, Phlomobacter, Candidatus Riesia, Candidatus Stammerula, Cedecea, Citrobacter, Cronobacter, Dickeya, Edwardsiella, Enterobacter, Erwinia, Escherichia, Ewingella, Grimontella, Hafnia, Klebsiella, Kluyvera, Leclercia, Leminorella, Margalefia, Moellerella, Morganella, Obesumbacterium, Pantoea, Pectobacterium, Photorhabdus, Phytobacter, Plesiomonas, Pragia, Proteus, Providencia, Rahnella, Raoultella, Salmonella, Samsonia, Serratia, Shigella, Sodalis, Tatumella, Thorasellia, Tiedjeia, Trabulsiella, Wigglesworthia, Xenorhabdus, Yersinia, and Yokenella. In preferred embodiments, the bacteria are E. coli.

C. Recombinant Polynucleotides Encoding Heterologous Genes

In certain embodiments of the present disclosure, the recombinant polynucleotide contains a nucleotide sequence encoding one or more heterologous genes having any function. For example, the heterologous genes may encode one or more enzymes involved in one or more biological pathways. Such pathways may include the alginate metabolic pathway, the cellulose/cellobiose metabolic pathway, the isoprenoid pathway, the fatty acid biosynthetic pathway, the isobutanol pathway, the n-butanol pathway, and the 2-butanol pathway.

Certain embodiments may also utilize recombinant unicellular organisms to enhance the efficiency of the pathway encoded by the heterologous genes. For instance, these organisms may be modified to enhance expression of endogenous genes which may positively regulate the heterologous pathway encoded by the recombinant polynucleotide of the present disclosure. One way of achieving this enhancement is to provide additional exogenous copies of such positive regulator genes. Similarly, negative regulators of the pathway, which are endogenous to the cell, may be removed.

In certain embodiments, the unicellular organism may be capable of producing an increased amount of a given product (e.g., ethanol) when containing the recombinant polynucleotide of the present disclosure. For example, E. coli that contains the recombinant polynucleotide may also be modified to produce an increased amount of ethanol as compared to E. coli that does not contain the recombinant polynucleotide. Thus, certain embodiments include an E. coli cell that contains the recombinant polynucleotide and that is capable of producing an increased amount of ethanol, such as by containing one or more genes encoding and expressing particular enzymes, including functional variants thereof. Examples of such genes are pyruvate decarboxylase (pdc) and two alcohol dehydrogenases (adhA and adhB) obtained from Zymomonas mobilis.

The recitation “polynucleotide” as used herein designates mRNA, RNA, cRNA, rRNA, cDNA, or DNA. The term typically refers to polymeric form of nucleotides of at least 10 bases in length, either ribonucleotides or deoxynucleotides or a modified form of either type of nucleotide. The term includes single and double stranded forms of DNA.

As will be understood by those skilled in the art, the nucleotide sequences of the present disclosure can include genomic sequences, extra-genomic and plasmid-encoded sequences, and smaller engineered gene segments that express, or may be adapted to express, proteins, enzymes, polypeptides, peptides, and the like. Such segments may be naturally isolated, or modified synthetically by the hand of man.

Polynucleotides may be single-stranded (coding or antisense) or double-stranded. Additional coding or non-coding sequences may, but need not, be present within a polynucleotide of the present disclosure, and a polynucleotide may, but need not, be linked to other molecules and/or support materials.

Polynucleotides may contain a native sequence (i.e., an endogenous sequence) or may contain a variant, or a biological functional equivalent of such a sequence. Polynucleotide variants may contain one or more substitutions, additions, deletions and/or insertions, as further described below, preferably such that the enzymatic activity of the encoded polypeptide is not substantially diminished relative to the unmodified polypeptide, and preferably such that the enzymatic activity of the encoded polypeptide is improved (e.g., optimized) relative to the unmodified polypeptide.

The polynucleotides of the present disclosure, regardless of the size of the coding sequence itself, may be combined with other DNA sequences, such as promoters, polyadenylation signals, additional restriction enzyme sites, multiple cloning sites, other coding segments, and the like, such that their overall size may vary considerably. It is contemplated that a polynucleotide of almost any size may be employed, with the total size preferably being limited by the ease of preparation and use in the intended recombinant DNA protocol, such as the polynucleotide-carrying capacity of a phage when phage delivery is used in the present disclosure. In some embodiments, the size of the polynucleotide is at least 1, 2, 3, 4, 5, 6, 7, 8, 9, 10, 11, 12, 13, 14, 15, 16, 17, 18, 19, 20, 30, 40, 50, 100 kb (including all integers and decimal points in between, e.g., 1.2, 1.3, 1.4, 1.5, 5.5, 5.6, 5.7, 60, 70, etc.). In some embodiments, the size of the recombinant polynucleotide is at least 11 kilobases. In some embodiments, the size of the recombinant polynucleotide is selected from A) at least 12 kilobases; B) at least 13 kilobases; or C) at least 14 kilobases.

The polynucleotides may be prepared, manipulated and/or expressed using any of a variety of well established techniques known and available in the art. For example, polynucleotide sequences that encode substantially the same or a functionally equivalent amino acid sequence may be produced and these sequences may be used to clone and express a given polypeptide.

As will be understood by those of skill in the art, it may be advantageous in some instances to use polypeptide-encoding polynucleotide sequences possessing non-naturally occurring codons. For example, codons preferred by a particular unicellular organism can be selected to increase the rate of protein expression or to produce a recombinant RNA transcript having desirable properties, such as a half-life which is longer than that of a transcript generated from the naturally occurring sequence. Such nucleotides are typically referred to as “codon-optimized.” Any of the nucleotide sequences described herein may be utilized in such a “codon-optimized” form. For example, the nucleotide coding sequence of an enzyme may be codon-optimized for expression in E. coli. Moreover, the nucleotide sequences of the present disclosure can be engineered using methods generally known in the art in order to alter polypeptide encoding sequences for a variety of reasons, including but not limited to, alterations which modify the cloning, processing, expression, and/or activity of the gene product.

In some embodiments, the recombinant polynucleotides containing nucleotide sequences encoding heterologous genes involved in biological pathways can be chemically synthesized from published sequences or obtained directly from host cells harboring the pathway (e.g., by cDNA library screening or PCR amplification). The genes may be included in an expression cassette. Such expression cassettes contain sequences that assist initiation and termination of transcription (e.g., promoters and terminators).

In certain embodiments, the nucleotide sequences encoding one or more heterologous genes polypeptides may be optimized. As used herein, “optimized” refers to the polynucleotide encoding a polypeptide having an altered biological activity, such as by the genetic alteration of the polynucleotide such that the encoded polypeptide had improved functional characteristics in relation to the wild-type polypeptide. Any of the recombinant polynucleotides described herein may optionally have one or more nucleotide sequences encoding for optimized polypeptides.

Typically, the improved functional characteristics of the polypeptide relate to the suitability of the polypeptide for use in a biological pathway (e.g., a metabolic pathway). Certain embodiments, therefore, contemplate the use of “optimized” biological pathways. An exemplary optimized nucleotide sequence may encode a polypeptide containing one or more alterations or mutations in its amino acid coding sequence (e.g., point mutations, deletions, addition of heterologous sequences) that facilitate improved expression and/or stability in a given unicellular organism, allow regulation of polypeptide activity in relation to a desired substrate (e.g., inducible or repressible activity), modulate the localization of the polypeptide within a cell (e.g., intracellular localization, extracellular secretion), and/or affect the polypeptide's overall level of activity in relation to a desired substrate (e.g., reduce or increase enzymatic activity). The encoded polypeptide may also be optimized for use with a particular unicellular organism, such as E. coli, by altering one or more pathways within that system or cell, such as by altering a pathway that regulates the expression (e.g., upregulation), localization, and/or activity of the optimized polypeptide, or by altering a pathway that minimizes the production of undesirable by-products, among other alterations. In this manner, a polypeptide may be optimized with or without altering its wild-type amino acid sequence or original chemical structure. Optimized polypeptides or biological pathways may be obtained, for example, by direct mutagenesis or by natural selection for a desired phenotype, according to techniques known in the art.

In certain embodiments, optimized nucleotide or polypeptide sequences may include a nucleotide coding sequence or amino acid sequence that is 50% to 99% identical to the nucleotide or amino acid sequence of a reference (e.g., wild-type) gene or polypeptide. In certain embodiments, an optimized polypeptide may have about 1, 2, 3, 4, 5, 6, 7, 8, 9, 10, 20, 30, 40, 50, 100 (including all integers and decimal points in between, e.g., 1.2, 1.3, 1.4, 1.5, 5.5, 5.6, 5.7, 60, 70, etc.), or more times the biological activity of a reference polypeptide.

D. Alginate Metabolism

In certain embodiments of the present disclosure, the one or more heterologous genes integrated into the genome is an alginate lyase, a DEHU reductase, and/or an alginate transporter and integration of the one or more heterologous genes modifies the unicellular organism to be able to grow on alginate-containing or alginate-derived media.

Alginate is a block co-polymer of β-D-mannuronate (M) and α-D-gluronate (G) (M and G are epimeric about the C5-carboxyl group). Each alginate polymer comprises regions of all M (polyM), all G (polyG), and/or the mixture of M and G (polyMG). A source of alginate is aquatic or marine biomass, which contains alginate as one of the main sugar components. Examples of aquatic or marine biomass include, but are not limited to, kelp, giant kelp, seaweed, algae, and marine microflora, microalgae, sea grass, and the like. Alginate can thus be extracted from various sources. In certain embodiments, alginate may be chemically degraded into its component monomers using chemical catalysts. Such embodiments may use, for example, formate, hydrochloric acid, sulfuric acid, in addition to other suitable acids known in the art as chemical catalysts.

The terms “alginate-containing or alginate-derived media” refer to growth media containing alginate or alginate-derived polysaccharides. These may include saturated polysaccharide, such as β-D-mannuronate, α-L-gluronate, dialginate, trialginate, pentalginate, hexylginate, heptalginate, octalginate, nonalginate, decalginate, undecalginate, dodecalginate and polyalginate, as well as unsaturated polysaccharides such as 4-deoxy-L-erythro-5-hexoseulose uronic acid, 4-(4-deoxy-beta-D-mann-4-enuronosyl)-D-mannuronate or L-guluronate, 4-(4-deoxy-beta-D-mann-4-enuronosyl)-dialginate, 4-(4-deoxy-beta-D-mann-4-enuronosyl)-trialginate, 4-(4-deoxy-beta-D-mann-4-enuronosyl)-tetralginate, 4-(4-deoxy-beta-D-mann-4-enuronosyl)-pentalginate, 4-(4-deoxy-beta-D-mann-4-enuronosyl)-hexylginate, 4-(4-deoxy-beta-D-mann-4-enuronosyl)-heptalginate, 4-(4-deoxy-beta-D-mann-4-enuronosyl)-octalginate, 4-(4-deoxy-beta-D-mann-4-enuronosyl)-nonalginate, 4-(4-deoxy-beta-D-mann-4-enuronosyl)-undecalginate, and 4-(4-deoxy-beta-D-mann-4-enuronosyl)-dodecalginate.

An exemplary source of alginate metabolic enzymes is Agrobacterium tumefaciens (A. tumefaciens) C58, which is able to metabolize relatively small sizes of alginate molecules (1,000-mers) as a sole source of carbon and energy. Since A. tumefaciens C58 has long been used for plant biotechnology, the genetics of this organism has been relatively well studied, and many genetic tools are available and compatible with other gram-negative bacteria such as E. coli. Thus, in certain embodiments, the recombinant polynucleotide contains a nucleotide sequence encoding alginate metabolic enzymes from this microbe.

Another exemplary source of alginate metabolic enzymes is Vibrio splendidus (V. splendidus), which is known to be able to metabolize alginate to support growth.

In certain embodiments of the present disclosure, the unicellular organism is capable of growing on alginate-containing or alginate-derived media by using alginate as a sole source of carbon may utilize a naturally-occurring or endogenous copy of a dehydratase, kinase, and/or aldolase. For instance, E. coli contains endogenous dehydratases, kinases, and aldolases that are capable of catalyzing the appropriate steps in the conversion of polysaccharides to a suitable monosaccharide. In certain embodiments, the naturally-occurring dehydratase or kinase may also be over-expressed, such as by providing an exogenous copy of the naturally-occurring dehydratase, kinase or aldolase operable linked to a highly constitutive or inducible promoter.

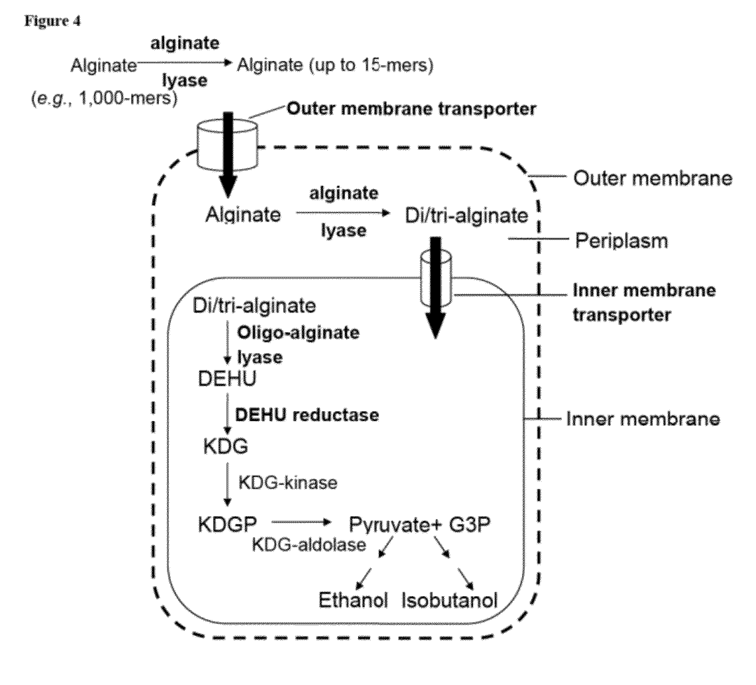

Certain embodiments may also utilize recombinant E. coli to enhance the efficiency of the KDG degradation pathway. For instance, in E. coli, KDG is a common metabolic intermediate in the degradation of hexuronates such as D-glucuronate and D-galacturonate and enters into Entner-Doudoroff pathway where it is converted to pyruvate and glyceraldehyde-3-phosphate (G3P) (FIG. 4). In this pathway, KDG is first phosphorylated by KDG kinase (KdgK) followed by its cleavage into pyruvate and glyceraldehyde-3-phosphate (G3P) using 2-keto-3-deoxy-D-6-posphate-gluconate (KDPG) aldolase (KdgA). The expression of these enzymes concurrently with KDG permease (e.g., KdgT) is negatively regulated by KdgR and is almost none at basal level. Hence, to increase the conversion of KDG to pyruvate and G3P, the negative regulator KdgR may be removed. To further improve the pathway efficiency, exogenous copies of KdgK and KdgA may also be incorporated into recombinant cells.

In certain embodiments, a unicellular organism which is able to grow on a polysaccharide (e.g., alginate or alginate-derived products) as a sole source of carbon may be capable of producing an increased amount of a given product (e.g., ethanol) while growing on that polysaccharide. For example, E. coli able to grow on alginate or alginate-derived media may also be modified to produce an increased amount of ethanol from alginate as compared to E. coli that is not able to grow on alginate. Thus, certain embodiments include E. coli that is capable of growing on alginate or alginate derivatives as a sole source carbon, and that is capable of producing an increased amount of ethanol, such as by containing one or more genes encoding and expressing a pyruvate decarboxylase (pdc) and/or an alcohol dehydrogenase, including functional variants thereof. Examples of such genes are pyruvate decarboxylase (pdc) and two alcohol dehydrogenases (adhA and adhB) obtained from Zymomonas mobilis.

(i). Alginate Lyases

In certain embodiments, the recombinant polynucleotide contains a nucleotide sequence encoding alginate lyases (ALs). ALs are mainly classified into two distinctive subfamilies depending on their acts of catalysis: endo- (EC 4.2.2.3) and exo-acting (EC 4.2.2.11) ALs. In some embodiments, the ALs include exo-acting ALs, e.g., M specific and G specific ALs or endo-acting ALs, which randomly cleave alginate via a 1-elimination mechanism and mainly depolymerizing alginate to di-, tri- and tetra-saccharides. The uronate at the non-reducing terminus of each oligosaccharide is converted to unsaturated sugar uronate, 4-deoxy-α-L-erythro-hex-4-ene pyranosyl uronates. The exo-acting ALs catalyze further depolymerization of these oligosaccharides and release unsaturated monosaccharides, which may be non-enzymatically converted to monosaccharides, including α-keto acid, 4-deoxy-α-L-erythro-hexoselulose uronate (DEHU). Certain embodiments may include endoM-, endoG-, and exo-acting ALs to degrade or depolymerize alginate to a monosaccharide such as DEHU.

Embodiments of the present disclosure may also include lyases such as alginate lyases isolated from various sources, including, but not limited to, marine algae, mollusks, and wide varieties of microbes such as genus Pseudomonas, Vibrio, and Sphingomonas. Many alginate lyases are endo-acting M specific, several are G specific, and few are exo-acting. For example, ALs isolated from Sphingomonas sp. strain Al include five endo-acting ALs, Al-I, Al-II, Al-II′, Al-III, and Al-IV′ and an exo-acting AL, Al-IV.

In addition to these ALs, exolytic AL Atu3025 derived from A. tumefaciens has high activity for depolymerization of oligo-alginate, and may be used in certain embodiments of the present disclosure. In certain embodiments, the recombinant polynucleotide may include the nucleotide sequences encoding Al-I, Al-II′, Al-IV, and Atu3025, and may include optimal codon usage for E. coli.

In certain embodiments, the AL is an oligo-alginate lyase. Certain examples of alginate lyases or oligo-alginate lyases that may be utilized herein include the oligo-alginate lyase Atu3025 isolated from A. tumefaciens. Certain examples of ALs that may be utilized herein include the alginate lyase enzymes shown in Table 1, as well as the secreted AL encoded by Vs24254 from V. splendidus.

Table 1 shows a list of alginate lyase genes/proteins that may be utilized in present disclosure.

| Protein | Organism | GenBank/GenPept | |
|---|---|---|---|
| Family 5 | | | |
| alginate lyase (AlgL) | Azotobacter chroococcum | AJ223605 | CAA11481.1 |
| | ATCC 4412 | | |
| alginate lyase (AlgL) | Azotobacter vinelandii | AF027499 | AAC04567.1 |
| | | AF037600 | AAC32313.1 |
| alginate lyase (Alg) | Cobetia marina N-1 | AB018795 | BAA33966.1 |
| alginate lyase (AlgL) | Pseudomonas aeruginosa 8830 | L14597 | AAA71990.1 |
| alginate lyase (AlgL) | Pseudomonas aeruginosa FRD1 | U27829 | AAA91127.1 |
| alginate lyase (AlgL; PA3547) | Pseudomonas aeruginosa PAO1 | AE004775 | AAG06935.1 |
| | | NC_002516 | NP_252237.1 |
| alginate lyase (AlgL) | Pseudomonas sp. QD03 | AY380832 | AAR23929.1 |
| alginate lyase (AlgL) | Pseudomonas sp. QDA | AY163384 | AAN63147.1 |
| alginate lyase (AlgL) | Pseudomonas syringae pv. | AF222020 | AAF32371.1 |
| | syringae FF5 | | |
| alginate lyase (aly; | Sphingomonas sp. A1 | — | 2009330A |
| A1-I/PolyG + PolyM; | | AB011415 | BAB03312.1 |
| A1-II/PolyG; A1-III/PolyM) | | | |
| Family 6 | | | |
| alginate lyase (AlyP) | Pseudomonas sp. OS-ALG-9 | D10336 | BAA01182.1 |
| Family 7 | | | |
| guluronate lyase (alyPG) | Corynebacterium sp. ALY-1 | AB030481 | BAA83339.1 |
| poly(-L-guluronate) lyase (AlyA | Klebsiella pneumoniae subsp. | L19657 | AAA25049.1 |
| | aerogenes | | |
| alginate lyase/poly- | Photobacterium sp. ATCC | X70036 | CAA49630.1 |
| mannuronate lyase (AlxM) | 43367 | | |
| alginate lyase (PA1167) | Pseudomonas aeruginosa PAO1 | AE004547 | AAG04556.1 |
| | | NC_002516 | NP_249858.1 |
| alginate lyase (A1-II′) | Sphingomonas sp. A1 | AB120939 | BAD16656.1 |
| alginate lyase (aly; | Sphingomonas sp. A1 | — | 2009330A |
| A1-I/PolyG + PolyM; | | AB011415 | BAB03312.1 |
| A1-II/PolyG; A1-III/PolyM) | | | |
| poly(a-L-guluronate) lyase | Vibrio halioticoli IAM14596T | AF114039 | AAF22512.1 |
| (AlyVGI; AlyVG1) | | | |
| alginate lyase/poly- | Vibrio sp. O2 | DQ235160 | ABB36771.1 |
| mannuronate lyase (AlyVOA) | | | |
| alginate lyase/poly- | Vibrio sp. O2 | DQ235161 | ABB36772.1 |
| mannuronate lyase (AlyVOB) | | | |
| alginate lyase (AlyVI) | Vibrio sp. QY101 | AY221030 | AAP45155.1 |
| exo-oligoalginate lyase | Haliotis discus hannai | AB234872 | BAE81787.1 |
| (HdAlex; HdAlex-1) | | | |
| alginate lyase (HdAly) | Haliotis discus hannai | AB110094 | BAC87758.1 |
| polysaccharide lyase acting on | Chlorella virus CVK2 | AB044791 | BAB19127.1 |
| glucuronic acid (vAL-1) | | | |
| alginate lyase (AlyII) | Pseudomonas sp. OS-ALG-9 | AB003330 | BAA19848.1 |
| Family 18 | | | |
| alginate lyase | Pseudoalteromonas sp. 272 | | |
| alginate lyase (Aly) | Pseudoalteromonas sp. | AF082561 | AAD16034.1 |
| | IAM14594 | | |
| Family 15 | | | |
| exotype alginate lyase | Agrobacterium tumefaciens str. | AE009232 | AAL43841.1 |
| (Atu3025) | C58 | NC_003305 | NP_533525.1 |
| exotype alginate lyase | Agrobacterium tumefaciens str. | AE008381 | AAK90358.1 |
| (AGR_L_3558p) | C58 (Cereon) | NC_003063 | NP_357573.1 |
| oligo alginate lyase (A1-IV) | Sphingomonas sp. A1 | AB011415 | BAB03319.1 |
| alginate lyase (A1-IV′) | Sphingomonas sp. A1 | AB176667 | BAD90006.1 |

(ii). DEHU Reductase

In certain embodiments, the recombinant polynucleotide contains a nucleotide sequence encoding a polypeptide that reduces 4-deoxy-L-erythro-5-hexoseulose uronate (DEHU) to a monosaccharide that is suitable for biofuel biosynthesis, such as 2-keto-3-deoxy-D-gluconate (KDG). Such exemplary polypeptides, include DEHU hydrogenases/reductases such as ADH1 through ADH12 enzymes isolated from A. tumefaciens C58 (see US patent application 2009/0139134).

As a further example, Pseudomonas grown using alginate as a sole source of carbon and energy contains a DEHU hydrogenase enzyme that uses NADPH as a co-factor, is more stable when NADP⁺ is present in the solution, and is active at ambient pH. Thus, certain embodiments as described herein may incorporate nucleotide sequences encoding DEHU reductase derived or obtained from various microbes, in which these microbes may be capable of growing on polysaccharides such as alginate as a source of carbon and/or energy.

(iii). Alginate Transporters

In certain embodiments, the recombinant polynucleotide contains a nucleotide sequence encoding a cellular component by which polysaccharides and macromolecules such as alginate polymers may be directly incorporated into the cytosol and degraded inside the unicellular organism. The transporters may be located on the inner membrane or the outer membrane of a cell. These transporters, such as one found in Sphingomonas sp. strain Al, may consist of a pit on the outer membrane (e.g., AlgR), alginate-binding proteins in the periplasm (e.g., AlgQ1 and Alg Q2), and an ATP-binding cassette (ABC) transporter (e.g., AlgM1, AlgM2, and AlgS). Incorporated polysaccharides such as alginate may then be readily depolymerized by lyases such as alginate lyases produced in the cytosol. Certain embodiments may incorporate genes encoding a transporter (e.g., ccpA, algS, algM1, algM2, algQ1, algQ2) to introduce this ability to the unicellular organism or gram-negative bacteria. Certain examples of alginate ABC transporters that may be utilized herein, include ABC transporters Atu3021, Atu3022, Atu3023, Atu3024, algM1, algM2, AlgQ1, AlgQ2, AlgS, OG2516_{—}05558, OG2516_{—}05563, OG2516_{—}05568, and OG2516_{—}05573 (OG refers to Oceanicola granulosus HTCC2516), including functional variants thereof. Certain examples of alginate symporters that may be utilized herein include symporters V12B01_{—}24239 and V12B01_{—}24194 from V. splendidus 12B01, among others, including functional variants thereof. One additional example of an alginate outer membrane transporter includes V12B01_{—}24269, and variants thereof.

In certain embodiments, the recombinant polynucleotide contains a nucleotide sequence encoding a cellular component which is able to transport monosaccharides (e.g., DEHU) and oligosaccharides from the media to the cytosol to efficiently utilize these monosaccharides as a source of carbon and/or energy. Certain embodiments may incorporate genes encoding monosaccharide permeases (i.e., monosaccharide transporters) such as DEHU permeases isolated from bacteria that grow on polysaccharides such as alginate as a source of carbon and/or energy. Other embodiments may also include redesigned native permeases or transporters with altered specificity for monosaccharide (e.g., DEHU) transportation. E. coli contains several permeases able to transport monosaccharides, which include, but are not limited to KdgT for 2-keto-3-deoxy-D-gluconate (KDG) transporter, ExuT for aldohexuronates such as D-galacturonate and D-glucuronate transporter, GntT, GntU, GntP, and GntT for gluconate transporter, and KgtP for proton-driven α-ketoglutarate transporter. Unicellular organisms described herein may contain any of these permeases, in addition to those permeases known to a person of skill in the art and not mentioned herein, and may also include permease enzymes redesigned to transport other monosaccharides, such as DEHU.

E. Cellulose/Cellobiose Metabolism

In certain embodiments of the present disclosure, the one or more heterologous genes integrated into the genome is an endo-type cellulase, an exo-type cellulase, a β-glucosidase, and/or a cellulose/cellobiose transporter, and integration of the one or more heterologous genes modifies the unicellular organism to be able to grow on cellulose/cellobiose-containing media.

Cellulose is the predominant polysaccharide in biomass (with others including hemicellulose, lignin, and pectin). It is a homopolymer of anhydrocellobiose (a linear β-(1-4)-D-glucan), and includes glucose units linked together in β-1,4-glycosidic linkages. Although generally polymorphous, cellulose is found in plant tissue primarily as an insoluble crystalline matrix of parallel glucan chains.

Purified celluloses include holocellulases, such as Solka Flok, microcrystalline celluloses, such as Avicel® and Sigmacell®, and the highly soluble cellulose ether, carboxymethylcellulose (CMC). In certain embodiments, the cellulose/cellobiose-containing media contains cellulose-containing substrates. Such cellulose-containing substrates include soluble and substrates, such as cellodextrins and their derivatives, short chain cellulase, β-methylumbelliferyl-oligosaccharides, p-nitrophenol-oligosaccharides, long chain cellulose derivatives, carboxymethyl cellulose (CMC), hydroxyethyl cellulose (HEC), and insoluble substrates, including cotton, Whatman No. 1 filter paper, Pulp (e.g., Solka Floc), crystalline cellulose, such as cotton, microcrystalline cellulose (e.g., Avicel®), Valonia cellulose, bacterial cellulose, amorphous cellulose (e.g., PASC, alkali-swollen cellulose), dyed cellulose, fluorescent cellulose, chromogenic and fluorephoric derivatives, such as trinitrophenyl-carboxymethylcellulose (TNP-CMC) and Fluram-cellulose, practical cellulose-containing substrates, α-cellulose, and pre-treated lignocellulosic biomass.

The cellulose/cellobiose metabolizing enzymes are of considerable current interest for converting the cellulosic content of biomass to fermentable sugars for biofuels production. Several enzymes (exo-type cellulase, endo-type cellulase, and β-glucosidase) act in concert to hydrolyze cellulose to glucose.

(i). Endo-Type Cellulase

In certain embodiments, the recombinant polynucleotide contains a nucleotide sequence encoding endo-type cellulases or endoglucanases (1,4-β-D-glucan 4-glucanohydrolases; EC 3.2.1.4) which break internal bonds to disrupt the crystalline structure of cellulose and expose individual cellulose polysaccharide chains. These enzymes yield cellobiose and cello-oligosaccharides as hydrolysis products. Examples of endo-type cellulases that may be utilized herein include but are not limited to Saccharophagus degradans (S. degradans) Sde_{—}2272 and Sde_{—}2636 and Clostridium cellulolyticum CelG, CelH, CelJ. Additional examples include all the enzymes in the group EC 3.2.1.4.

(ii). Exo-Type Cellulose

In certain embodiments, the recombinant polynucleotide contains a nucleotide sequence encoding exo-type cellulases or exoglucanases, which include cellodextrinases (1,4-β-D-glucan glucanohydrolases; EC 3.2.1.74) and cellobiohydrolases (CBHs) (1,4-β-D-glucan cellobiohydrolases; EC 3.2.1.91). These enzymes cleave two to four units from the ends of the exposed chains produced by endocellulases (either from the reducing end or the non-reducing end), resulting in tetrasaccharides or disaccharides such as cellobiose. Examples of exo-type cellulases that may be utilized herein include but are not limited to Clostridium cellulolyticum Ccel_{—}0374, Cellvibrio japonicas CelC and CelD, Clostridium thermocellum CbhA, CelK, and CelO, and Trichoderma CbhII. Additional examples include all the enzymes in EC 3.2.1.74 and EC 3.2.1.91.

(iii). β-Glucosidase

In certain embodiments, the recombinant polynucleotide contains a nucleotide sequence encoding a β-glucosidase (β-glucoside glucohydrolases; EC 3.2.1.21) which hydrolyzes the exo-cellulase product into individual monosaccharides. Examples of β-glucosidases that may be utilized herein include but are not limited to Pyrococcus furiosus PF0442, PF0073, and PF0132, Clostridium thermocellum BglA, and Arabidopsis thaliana Bglu18, Bglu21, Bglu22, Bglu23, Bglu25, and Bglu44-46. Additional examples include all the enzymes in EC 3.2.1.21.

(iv). Cellulose/Cellobiose Transporters

In certain embodiments, the recombinant polynucleotide contains a nucleotide sequence encoding a cellular component by which polysaccharides and macromolecules, such as cellulose and/or cellobiose, may be directly incorporated into the cytosol and degraded inside the unicellular organism. The transporters may be located on the inner membrane or the outer membrane of a cell. Examples of cellobiose transporters include Sde_{—}1395 from Saccharophagus degradans 2-40, CebE found in Streptomyces reticuli (Schlosser et al. 1999), and CbtA found in Pyrococcus furiosus (Koning et al., 2001), both of which fall under the general class of ABC transporters. In addition to cellobiose, the latter has also been found to bind a multitude of substrates, including cellotriose, cellotetraose, cellopentaose, laminaribiose, laminaritriose, and sophorose. Polysaccharides incorporated into the cell via these transporters may then be readily depolymerized by cellulases such as β-glucosidase produced intracellularly.

F. Methods of Producing Commodity Chemicals

The present disclosure also provides methods of producing commodity chemicals, in particular, ethanol, n-butanol, isobutanol, and 2-butanol, by using the Cre-lox recombination system as described.

The present disclosure provides a method of producing a commodity chemical by: A) providing a unicellular organism having a genome containing a first lox site and a second lox site integrated in the genome of the unicellular organism where the first lox site contains a different sequence from the second lox site such that the first and second lox sites are incapable of recombining with each other; B) transforming the unicellular organism with a first plasmid and a second plasmid, where the first plasmid includes a recombinant polynucleotide containing a nucleotide sequence encoding one or more heterologous genes, where the recombinant polynucleotide is bounded by a third lox site and a fourth lox site where the third lox site has the same sequence as the first lox site and the fourth lox site has the same sequence as the second lox site, and where the second plasmid encodes Cre recombinase; C) culturing the unicellular organism under conditions such that Cre recombinase is expressed where Cre recombinase expression results in homologous recombination between the first and third lox sites and between the second and fourth lox sites and integration of the recombinant polynucleotide into the genome of the unicellular organism in between the first and second lox sites; D) growing the unicellular organism in media and under conditions where the one or more heterologous genes is expressed and a commodity chemical is produced, and E) collecting the commodity chemical.

The present disclosure also provides a method of producing a commodity chemical by: A) providing a unicellular organism having a genome containing a first lox site and a second lox site integrated in the genome of the unicellular organism where the first lox site contains a different sequence from the second lox site such that the first and second lox sites are incapable of recombining with each other, and having a plasmid encoding Cre recombinase; B) providing a donor cell containing a recombinant polynucleotide having a nucleotide sequence encoding one or more heterologous genes, where the recombinant polynucleotide is bounded by a third lox site and a fourth lox site where the third lox site has the same sequence as the first lox site and the fourth lox site has the same sequence as the second lox site; C) infecting the donor cell with a phage such that phage particles having the recombinant polynucleotide are produced and released from the donor cell; D) culturing the unicellular organism such that Cre recombinase is expressed; E) infecting the unicellular organism expressing Cre recombinase with the phage particles where Cre recombinase expression results in homologous recombination between the first and third lox sites and between the second and fourth lox sites and integration of the recombinant polynucleotide into the genome of the unicellular organism in between the first and second lox sites; F) growing the unicellular organism under conditions wherein the one or more heterologous genes is expressed and a commodity chemical is produced, and G) collecting the commodity chemical.

In certain embodiments, the recombinant polynucleotide can be of any size. In some embodiments, the size of the polynucleotide is at least 1, 2, 3, 4, 5, 6, 7, 8, 9, 10, 11, 12, 13, 14, 15, 16, 17, 18, 19, 20, 30, 40, 50, 100 kb (including all integers and decimal points in between, e.g., 1.2, 1.3, 1.4, 1.5, 5.5, 5.6, 5.7, 60, 70, etc.). In some embodiments, the recombinant polynucleotide is at least 11 kilobases in size. In some embodiments, the size of the recombinant polynucleotide is selected from A) at least 12 kilobases; B) at least 13 kilobases; or C) at least 14 kilobases.

In some embodiments where phage is used, the phage is P1vir. Other exemplary phages are as described in previous sections.

In some embodiments, the unicellular organism is yeast. In some embodiments, the unicellular organism is gram-negative bacteria, such as E. coli. Other exemplary unicellular organisms are as described in previous sections.

Exemplary recombinant polynucleotides encoding one or more heterologous genes are as described in previous sections.

In some embodiments, the one or more heterologous genes integrated into the genome is an alginate lyase, a DEHU reductase, and/or an alginate transporter and media contains, or is derived from, alginate. Exemplary alginate lyases, DEHU reductases, and alginate transporters are as described in previous sections.

In some embodiments, the one or more heterologous genes integrated into the genome is an endo-type cellulase, an exo-type cellulase, a β-glucosidase, and/or a cellulose/cellobiose transporter and the media contains cellulose/cellobiose. Exemplary endo-type cellulases, exo-type cellulases, β-glucosidases, and cellulose/cellobiose transporters are as described in previous sections.

In certain embodiments, the commodity chemical is ethanol. In some embodiments, the commodity chemical is isobutanol. In some embodiments, the commodity chemical is n-butanol. In other embodiments, the commodity chemical is 2-butanol.

### II. Unicellular Organisms Having Integrated Recombinant Polynucleotides in Genomes

The present disclosure provides unicellular organisms containing recombinant polynucleotides stably integrated into the genome where the recombinant polynucleotides contain nucleotide sequences encoding one or more heterologous genes. In certain embodiments, the recombinant polynucleotide can be of any size. In some embodiments, the size of the polynucleotide is at least 1, 2, 3, 4, 5, 6, 7, 8, 9, 10, 11, 12, 13, 14, 1506, 17, 18, 19, 20, 30, 40, 50, 100 kb (including all integers and decimal points in between, e.g., 1.2, 1.3, 1.4, 1.5, 5.5, 5.6, 5.7, 60, 70, etc.). In some embodiments, the recombinant polynucleotide is at least 11 kilobases in size. In some embodiments, the size of the recombinant polynucleotide is selected from A) at least 12 kilobases; B) at least 13 kilobases; or C) at least 14 kilobases.

In certain embodiments, the recombinant polynucleotide is stably integrated into the genome of the unicellular organism via plasmid delivery. In other embodiments, the recombinant polynucleotide is stably integrated into the genome of the unicellular organism via bacteriophage delivery. Exemplary phages are as described in previous sections.

In preferred embodiments, the recombinant polynucleotide is positioned between two lox sites in the genome of the unicellular organism.

In some embodiments, the unicellular organism is yeast. In some embodiments, the unicellular organism is gram-negative bacteria, such as E. coli. Other exemplary unicellular organisms are as described in previous sections.

Exemplary recombinant polynucleotides encoding one or more heterologous genes are as described in previous sections.

In some embodiments, the one or more heterologous genes integrated into the genome is an alginate lyase, a DEHU reductase, and/or an alginate transporter and the integration of the one or more heterologous genes into the genome modifies the unicellular organism to be able to grow on alginate-containing or alginate-derived media. Exemplary alginate lyases, DEHU reductases, and alginate transporters are as described in previous sections.

In some embodiments, the one or more heterologous genes integrated into the genome is an endo-type cellulase, an exo-type cellulase, a β-glucosidase, and/or a cellulose/cellobiose transporter and the integration of the one or more heterologous genes into the genome modifies the unicellular organism to be able to grow on cellulose/cellobiose-containing media. Exemplary endo-type cellulases, exo-type cellulases, β-glucosidases, and cellulose/cellobiose transporters are as described in previous sections.

In certain embodiments, the unicellular organism produces a commodity chemical, such as ethanol, isobutanol, n-butanol, and 2-butanol.

### EXAMPLES

### Example 1

### Genomic Insertion of lox Targeting Cassette

The use of Cre-lox recombination method for fragment delivery enabled recombination of a polynucleotide into a precise and predetermined location within the bacterial chromosome. The first step in the process was to insert lox sites into the host genome.

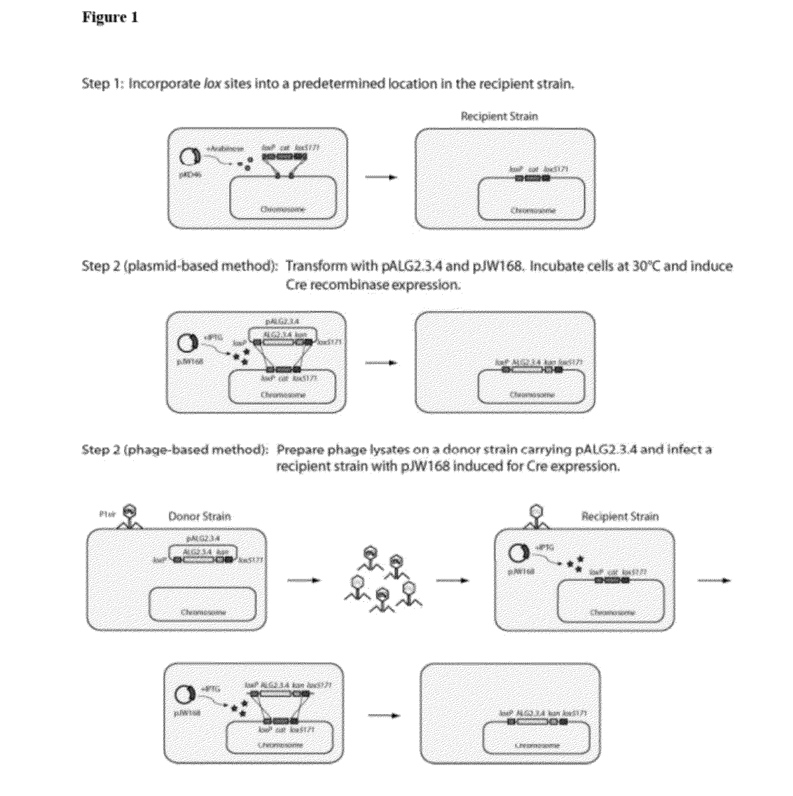

Lox sites were integrated into the ldhA locus of E. coli ATCC 8739 ΔldhA Δfrd::adhB Δpta::pdc ΔfocA-pflB::pdc-adhB (henceforth known as BAL1075, Table 2) using λ-RED recombination (Datsenko & Wanner, 2000) and a chloramphenicol marker (cat) (Step 1 of FIG. 1). The chloramphenicol resistance gene (cat) in the construct was flanked by two lox sites: loxP and lox5171 sites (Lee & Saito, 1998). Briefly, cat was amplified from pCm-R6K with primers CS001 lox5171-Cm sense, CS002 loxP-Cm anti, and Phusion Hot-Start II DNA polymerase (New England BioLabs, Ipswich, Mass.) (Table 3).

Two distinct and mutually exclusive lox sites (loxP, lox5171) (Lee & Saito, 1998) were incorporated into the primer sequences to allow for double-crossover recombination of similarly lox-flanked fragments into the genome. Although loxP and lox5171 sequences differed by only 2 base pairs (bp), they exhibited little to no cross-reactivity (Lee & Saito, 1998), making them particularly well-suited for mediating the necessary double crossover recombination events. In addition, the primers contained 28-29 bp homology with the ldhA region of E. coli ATCC 8739. The resulting ldhA::loxP-cat-lox5171 cassette was then re-amplified with primers CS003 lox-Cm sense 2 and CS004 lox-Cm anti 2 (Table 3) to extend its ldhA homology region (for a total of 78 bp) and subsequently utilized for λ-RED recombination as described in previously published protocols (Datsenko & Wanner, 2000).

Lox sites were integrated into the intergenic regions of gidB and atpI and mraZ and fruR through a similar method as above. The int(gidB-atpI)::loxP-cat-lox5171 integration cassette was amplified with CS095 lox5171-gidB-atpI sense and CS096 loxP-gidB-atpI anti, followed by a second round of amplification with CS097 lox-gidB-atpI sense 2 and CS098 lox-gidB-atpI anti 2 (Table 3). The int(mraZ-fruR)::loxP-cat-lox5171 cassette was amplified with CS105 lox5171-mraZ-fruR sense and CS106 loxP-mraZ-fruR anti, followed by a second round of amplification with CS107 lox-mraZ-fruR sense 2 and CS108 lox-mraZ-fruR sense 2 (Table 3). Colony selection was performed on LB-agar plates with 25 μg/mL chloramphenicol.

Correct integration events were verified by colony PCR and sequencing.

A schematic demonstrating the lox site integration process is shown in Step 1 of FIG. 1 and the primers used in this process as well as in the processes discussed in the Examples below are shown in Table 3.

Table 2 shows strains used in this study.

| Strains | Genotype |
|---|---|
| BAL847 | E. coli ATCC 8739 ΔldhA ΔpflB-focA |
| | Δfrd pALG7.8 pTrc-Zmpdc-ZmadhB |
| BAL1075 | E. coli ATCC 8739 ΔldhA Δfrd::ZmadhB |
| | Δpta::Zmpdc ΔpflB-focA::Zmpdc-ZmadhB |
| BAL1301 | E. coli ATCC 8739 ΔldhA ΔpflB-focA |
| | Δfrd::pF30- Zmpdc-ZmadhB |
| BAL1302 | E. coli ATCC 8739 ΔldhA ΔpflB-focA |
| | Δfrd::pH22- Zmpdc-ZmadhB |
| BAL1303 | E. coli ATCC 8739 ΔldhA ΔpflB-focA |
| | Δfrd::pG25- Zmpdc-ZmadhB |
| BAL1304 | E. coli ATCC 8739 ΔldhA ΔpflB-focA |
| | Δfrd::pJ5- Zmpdc-ZmadhB |
| BAL1373 | E. coli ATCC8739 ΔldhA ΔpflB-focA |
| | Δfrd::pG25- Zmpdc-ZmadhB pALG2.3 + N455-SM0524 |
| BAL1450 | E. coli ATCC8739 ΔldhA ΔpflB-focA Δfrd::pG25- |
| | Zmpdc-ZmadhB int(mraZ-fruR)::ALG2.3.4 N455-SM0524 |
| BAL1810 | E. coli ATCC8739 ΔldhA ΔpflB-focA |
| | Δfrd pALG2.3 + N455-SM024 pTrc-Zmpdc-ZmadhB |

Table 3 shows sequences of primers used in this study.

| Primer Name | Primer Sequence (5′ → 3′) |
|---|---|
| CS001 lox5171-Cm | CCAGATTGCTTAAGTTTTGCAGCGTAGTCATAACTTCGTATA |
| sense | GTACACATTATACGAAGTTAT |
| | TCGGCACGTAAGAGGTTCCAACTTT |
| | (SEQ ID NO: 1) |
| CS002 loxP-Cm anti | TACGACAAGAAGTACCTGCAACAGGTGAATAACTTCGTATA |
| | ATGTATGCTATACGAAGTTAT |
| | GGCGTTTAAGGGCACCAATAACTGC |
| | (SEQ ID NO: 2) |
| CS003 lox-Cm sense | CCTGGGTTGCAGGGGAGCGGCAAGATTAAACCAGTTCGTTC |
| 2 | GGGCAGGTTTCGCCTTTTT |
| | CCAGATTGCTTAAGTTTTGCAGCGTAGTC |
| | (SEQ ID NO: 3) |
| CS004 lox-Cm anti 2 | ATGTGATTCAACATCACTGGAGAAAGTCTTATGAAACTCGC |
| | CGTTTATAGCACAAAACAG |
| | TACGACAAGAAGTACCTGCAACAGGTGA |
| | (SEQ ID NO: 4) |
| CS005 ldhA verif | GCATGGGTAGTTAATATCCTGATTTAGCGA |
| sense | (SEQ ID NO: 5) |
| CS008 ldhA verif anti | GAAAGGTCATTGGGGATTATCTGAATCA |
| | (SEQ ID NO: 6) |
| CS009 Cm/Km hom | CTGACCGTTCTGTCCGTCACTTCCC |
| sense | (SEQ ID NO: 7) |
| CS010 Cm anti-hom | CTTAATCGCTGGCTTTTCTTCTTTCAAATCAATTCATTTAAAT |
| | AAGAGCCGAGTACTTAAGGCGTTTAAGGGCACCAATAACTG |
| | C |
| | (SEQ ID NO: 8) |
| CS011 lox5171- | CCCTGGGCCAACTTTTGGCGAAAATGAGACGTTGAATAACT |
| pKD13 anti | TCGTATAGTACACATTATACGAAGTTATATCCGTCGACCTGC |
| | AGTTCGA |
| | (SEQ ID NO: 9) |
| CS012 lox5171- | TGACGGAAGATCACTTCGCAGAATAAATAAATCCTGGTGTC |
| pKD13 anti 2 | CCTGTTGATACCGGGAAGCCCTGGGCCAACTTTTGGCGA |
| | (SEQ ID NO: 10) |
| CS013 hom1-pKD13 | CTTAATCGCTGGCTTTTCTTCTTTCAAATCAATTCATTTAAAT |
| sense | AAGAGCCGAGTACTTAATGTGTAGGCTGGAGCTGCTTCGA |
| | (SEQ ID NO: 11) |
| CS014 hom2-pKD13 | CAATGAACTCTTTCTTTATCCTAGATGAAAATCCATGGGAAG |
| sense | AACTTGGTGGCGGCATTATGTGTAGGCTGGAGCTGCTTCGA |
| | (SEQ ID NO: 12) |
| CS015 hom3-pKD13 | GATTTAGAATACTGAGAGGTGAAAAATCCCGGCTGTCGCAT |
| sense | AACTACTTGTCAGGTACAGTGTGTAGGCTGGAGCTGCTTCG |
| | A |
| | (SEQ ID NO: 13) |
| CS016 hom4-pKD13 | CAAGAACAACGCAGAAAAGCCACTCTAAACTCGACAGTTAT |
| sense | TGAGTGGCCTTCAGATCAATGTGTAGGCTGGAGCTGCTTCG |
| | A |
| | (SEQ ID NO: 14) |
| CS017 hom5-pKD13 | ACTCTAGTGCTAATTGTCATTCTGTCTTTCTACTGATCCAGC |
| sense | CCTCTCAAAGCCTGTATCTGTGTAGGCTGGAGCTGCTTCGA |
| | (SEQ ID NO: 15) |
| CS037 hom-pKD13 | GGCCAGTGAATTGTAATACGACTCACTATAGGGCGAATGTG |
| sense | TAGGCTGGAGCTGCTTCGA |
| | (SEQ ID NO: 16) |
| CS038 hom-loxP- | ATTAATGTGACCTTGGTATCAATGAGGGTGTACGTATAACTT |
| pKD13 anti | CGTATAGCATACATTATACGAAGTTATATCCGTCGACCTGCA |
| | GTTCGA |
| | (SEQ ID NO: 17) |
| CS039 pKD13 sense | TTTCCCAGTCACGACGTTGTAAAACGACGGCCAGTGAATTG |
| 2 | TAATACGACTCACTATAGG |
| | (SEQ ID NO: 18) |
| CS040 lox-pKD13 | GTTTTATATGAGTTTTAAGATGAACTTGGTATTAATGTGACC |
| anti 2 | TTGGTATCAATGAGGGTG |
| | (SEQ ID NO: 19) |
| CS095 lox5171-gidB- | GATGCCTTTGCAAGTTTATGATATTTCAGTCATAACTTCGTA |
| atpI sense | TAGTACACATTATACGAAGTTATTCGGCACGTAAGAGGTTC |
| | CAACTTT |
| | (SEQ ID NO: 20) |
| CS096 loxP-gidB- | TGTTCACTCTTTTGCATCAACAAGATAACGATAACTTCGTAT |
| atpI anti | AATGTATGCTATACGAAGTTATGGCGTTTAAGGGCACCAAT |
| | AACTGC |
| | (SEQ ID NO: 21) |
| CS097 lox-gidB-atpI | CGCACAGCATATTTATTTACTTGGCAAATGATGCCTTTGCAA |
| sense 2 | GTTTATGATATTTCAGTC |
| | (SEQ ID NO: 22) |
| CS098 lox-gidB-atpI | TACTGATATAACTGGTTACATTTAACGCCATGTTCACTCTTT |
| anti 2 | TGCATCAACAAGATAACG |
| | (SEQ ID NO: 23) |
| CS099 gidB-atpI | GGTCGAATCAGTGGTTAAACTTCAGGTTC |
| verif sense | (SEQ ID NO: 24) |
| CS100 gidB-atpI | GTTTCGACTCACGAGCGACACAGA |
| verif anti | (SEQ ID NO: 25) |
| CS105 lox5171- | GGTTAACAGTCCCTGTTGCGTCTGTGTGGCGATAACTTCGTA |
| mraZ-fruR sense | TAGTACACATTATACGAAGTTATTCGGCACGTAAGAGGTTC |
| | CAACTTT |
| | (SEQ ID NO: 26) |
| CS106 loxP-mraZ- | CGGGACTGGACATCAATATGCTTAAAGTAAAATAACTTCGT |
| fruR anti | ATAATGTATGCTATACGAAGTTATGGCGTTTAAGGGCACCA |
| | ATAACTGC |
| | (SEQ ID NO: 27) |
| CS107 lox-mraZ-fruR | CGACGCGAGCGGCATTTTAGGACATATCTTCCCCGGTTAAC |
| sense 2 | AGTCCCTGTTGCGTCTGTG |
| | (SEQ ID NO: 28) |
| CS108 lox-mraZ-fruR | CGCCAGGTGAATTTCCCTCTGGCGCGTAGAGTACGGGACTG |
| anti 2 | GACATCAATATGCTTAAAG |
| | (SEQ ID NO: 29) |
| CS109 mraZ-fruR | GTGTCAGTTTGCGACGCGAGC |
| verif sense | (SEQ ID NO: 30) |
| CS110 mraZ-fruR | GCGTAAGCCAAAACCTGGTTTAACG |
| verif anti | (SEQ ID NO: 31) |
| CS113 hom-pKD13 | TTAATTGCGTTGCGCTCACTGCCCGCTTTCCAGTCGGGAAAC |
| sense | CTGTCGTGCCAGCTGCAT TGTGTAGGCTGGAGCTGCTTCGA |
| | (SEQ ID NO: 32) |
| CS115 hom-loxP- | CGTTCATTTCACTTCATTTGCCGCATCTCACATTATAACTTCG |
| pKD13 anti | TATAGCATACATTATACGAAGTTAT |
| | ATCCGTCGACCTGCAGTTCGA |
| | (SEQ ID NO: 33) |
| CS116 lox-pKD13 | TTCTGCGTTGTTCTTGAGTCTAACTCTACGTAATATCCGTTC |
| anti 2 | ATTTCACTTCATTTGCCG |
| | (SEQ ID NO: 34) |
| CS120 hom-pKD13 | AATAATCTAAGATAATTACTGTCCTAATTTTCTAAGACCTAA |
| sense | ACAAAAGCCAGCTTAATC TGTGTAGGCTGGAGCTGCTTCGA |
| | (SEQ ID NO: 35) |

*The underlined sequences refer to the lox site (also underlined in the primer name) in each primer.

### Example 2

### Modification of pALG Plasmids for Cre-lox Recombination

The second step was to incorporate a complete alginate metabolic pathway (FIG. 4) into the E. coli BAL1075 (Table 2) strain using the Cre-lox recombination system. To achieve that goal, several plasmids (Tables 4 and 5) were constructed as follows.

(i) Construction of pALG2.3.4-8

Two lox sites—loxP and lox5171—were introduced into pALG2.3 (Tables 4 and 5) through four rounds of sequential modifications with λ-RED recombination (Datsenko & Wanner, 2000). pALG2.3.1 was constructed by amplifying cat from pCm-R6K with primers CS009 Cm/Km horn sense and CS010 Cm anti-horn (Table 3) and transforming the resulting cassette into DH5α pALG2.3 pKD46 electrocompetent cells. This initial step was necessary to change the antibiotic resistance of pALG2.3 from kanamycin to chloramphenicol (kan to cat) in order to facilitate downstream integration steps. To insert a loxP site into pALG2.3.1 (to form pALG2.3.2), the loxP::kan^{FRT} cassette was amplified from pKD13 with primers CS037 hom-pKD13 sense and CS038 hom-loxP-pKD13 anti (Table 3), followed by a second round of amplification with CS039 pKD13 sense 2 and CS040 lox-pKD13 anti 2 (Table 3). The kanamycin resistance gene (kan) allowed selection for proper integration events while the flanking FLP recognition target (FRT) sequence allowed excision of kan through expression of the FLP recombinase (Datsenko & Wanner, 2000). The kan excision, which allowed for the generation of marker-less integration strains capable of undergoing additional rounds of modification, was mediated by transformation with the FLP recombinase-expressing plasmid pCP20 as described in the literature (Datsenko & Wanner, 2000) to form pALG2.3.3. In the final step of modification, a lox5171 site was introduced into pALG2.3.3 by amplifying a lox5171::kan^{FRT} cassette from pKD13 with primers CS011 lox5171-pKD13 anti and CS013 hom1-pKD13 sense, followed by a second round of amplification with CS012 lox5171-pKD13 anti 2 and CS013 hom-pKD13 sense (Table 3). The cassette was subsequently transformed into DH5α pALG2.3.3 pKD46 to form pALG2.3.4, a plasmid containing a 35 kb fragment flanked by loxP, lox5171, and kan^{FRT} (Table 4).

Plasmids pALG2.3.5-8 were additionally constructed to provide integration cassettes of varying lengths, ranging from 6 kb to 27 kb (Table 4). lox5171::kan^{FRT} cassettes for construction were amplified with CS011 lox5171-pKD13 and CS014-17 hom2-5 pKD13 sense, followed by a second round of amplification with CS012 lox5171-pKD13 anti 2 and the same set of second primers (Table 3). Cassettes were subsequently transformed into DH5α pALG2.3.3 pKD46.

(ii) Construction of pALG2.5.4

Two lox sites—loxP and lox5171—were introduced into pALG3.0 to yield pALG2.5.4 through a similar method as described above (Tables 4 and 5). CS037 hom-pKD13 sense-CS115 hom-loxP-pKD13 anti and CS039 pKD13 sense 2-CS116 lox-pKD13 anti 2 primer pairings (Table 3) were used to amplify the loxP::kan^{FRT} cassette from pKD13 for the construction of pALG3.0.2. Excision of FRT-flanked kan was mediated by transformation with pCP20 to form pALG3.0.3, and the lox5171::kan^{FRT} cassette was amplified from pKD13 with primer pairs CS011 lox5171-pKD13 anti-CS120 hom-pKD13 sense and CS012 lox5171-pKD13 anti 2-CS120 hom-pKD13 sense (Table 3) to form pALG2.5.4 (Table 4).

(iii) Construction of pALG7.8.4

Two lox sites—loxP and lox5171 —were introduced into pALG7.8 to yield pALG7.8.4 (Tables 4 and 5). Construction of pALG7.8.2 and pALG7.8.3 proceeded through the same steps as for pALG2.3.2 and pALG2.3.3. The lox5171::kan^{FRT} cassette was amplified from pKD13 with primer pairs CS011 lox5171-pKD13 anti-CS113 hom-pKD13 sense and CS012 lox5171-pKD13 anti 2-CS113 hom-pKD13 sense (Table 3) to form pALG7.8.4 (Table 4).

Correct integration events were verified after each step in construction of the pALG plasmids by colony PCR and sequencing.

Table 4 shows the pALG plasmid versions and their corresponding lox-flanked cassette sizes.

| Original Plasmid | Modified Plasmid | Cassette Size (kb) |
|---|---|---|
| pALG2.3 | pALG2.3.4 | 35.3 |
| pALG2.3 | pALG2.3.5 | 26.5 |
| pALG2.3 | pALG2.3.6 | 19.2 |
| pALG2.3 | pALG2.3.7 | 10.3 |
| pALG2.3 | pALG2.3.8 | 5.6 |
| pALG3.0 | pALG2.5.4 | 28 |
| pALG7.8 | pALG7.8.4 | 59.1 |

Table 5 shows the genes contained in the pALG plasmids.

| Plasmid | Modifications added to the previous version of pALG plasmid* |
|---|---|
| pALG1.5 | Original fosmid clone isolated from genomic library of V. splendidus 12B01 |
| pALG1.6 | V12B01_24254 (alginate lyase) andV12B01_24259 (alginate lyase) added to pALG1.5 |
| pALG1.7 | V12B01_24264 (alginate lyase), V12B01_24269 (outer membrane transporter), and |
| | V12B01_24274 (alginate lyase) added to pALG1.6 |
| pALG2.3 | V12B01_24309 (outer membrane transporter) and V12B01_24324 (inner membrane transporter) added to pALG1.7 |
| pALG2.5 | V12B01_24309 (outer membrane transporter), V12B01_24324 (inner membrane transporter), and |
| | V12B01_24269 (outer membrane transporter) added to pALG1.5 |
| pALG3.0 | Atu_3020, Atu_3021, Atu_3022, Atu_3023, Atu_3024 (21-24: ABC transporter), Atu_3025 |
| | (oligo-alginate lyase), and Atu_3026 (DEHU reductase/hydrogenase) added to pALG2.5 |
| pALG3.5 | V12B01_24254 (alginate lyase) and V12B01_24259 (alginate lyase) added to pALG3.0 |
| pALG4.0 | Sde_3602 (Glutathione synthetase), Sde_3603 (β-glucosidase 1A: Bgl1A), Sde_1394 (β- |
| | glucosidase 1B: Bgl1B), Sde_1395 (cellobiose transporter), Sde_2674 (β-glucosidase 3C: |
| | Bgl3C), Sde_2637 (tRNA pseudouridine synthase B), and Atu_3019 were added to the pALG3.5 |
| pALG7.2 | P_{H207}-Ag43-ΔPaAly (alginate lyase) added to pALG4.0 |
| pALG7.8 | V12B01_24264-24274 added to pALG7.2 |

*Atu indicates genes from A. tumefaciens; Sde indicates genes from S. degradans; P_{H207} and Ag43 refers to a promoter from Coliphage and a carrier protein from E. coli respectively; ΔPaALY refers to alginate lyase from Pseudoalteromonas sp. SM0524

### Example 3

### Plasmid-Based Cre-lox Recombination

In the second step, the alginate metabolic pathway (FIG. 4) was integrated into the host genome via Cre-lox recombination. The alginate metabolic pathway was provided on a single-copy plasmid, such as pALG2.3.4, described above (Table 4). A schematic demonstrating the process is shown in Step 2 of FIG. 1.

BAL1075 ldhA::loxP-cat-lox5171 was transformed with pALG2.3.4 (Table 4) and pJW168 (Lucigen, Middleton, Wis.), a plasmid containing a temperature-sensitive replicon and an inducible Cre recombinase. After overnight growth in Luria-Bertani (LB) medium at 30° C., 25 μL was used to inoculate 2.5 mL fresh LB with 1 mM isopropy-β-D-thiogalactopyranoside (IPTG) and 12.5 μg/mL kanamycin. Cultures were grown for 3-6 hours at 30° C. and streaked out on LB-agar plates with kanamycin to isolate single colonies. After overnight growth at 37° C., individual colonies were streaked out on LB-kanamycin and LB-chloramphenicol (25 μg/mL chloramphenicol) to identify chloramphenicol-sensitive colonies. It was hypothesized that strain that have successfully undergone fragment integration into the targeted locus would exhibit chloramphenicol-sensitivity due to cassette replacement.

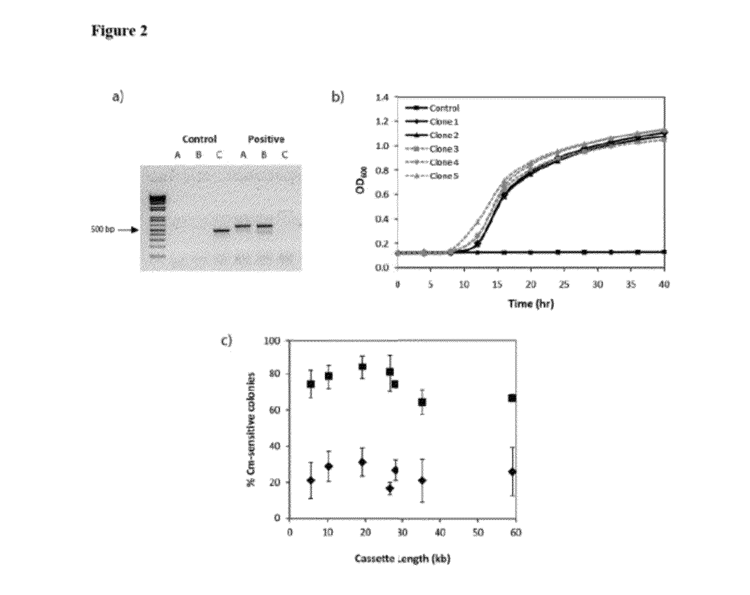

The colonies were additionally verified for proper end integration by colony PCR using primer pairs CS005 ldhA verif sense-CS063 left verif anti and CS008 ldhA verif anti-CS064 right verif sense (Table 3). The results for the verification of IdhA junction, shown in FIG. 2 (a), revealed correct fragment placement in all 10 strains tested.

Correct colonies were also streaked out on LB-ampicillin plates to verify loss of pJW168. The concentration of ampicillin was 100 μg/mL.

A similar procedure was used for integration into the strains BAL1075 int(gidB-atpI)::loxP-cat-lox5171 and BAL1075 int(mraZ-fruR)::loxP-cat-lox5171. Primers used for colony PCR were as follows: int(gidB-atpI)—CS063 left verif anti-CS099 gidB-atpI verif sense and CS064 right verif sense-CS100 gidB-atpI verif int(mraZ-fruR)—CS064 right verif sense—CS109 mraZ-fruR verif sense and CS063 left verif anti-CS110 mraZ-fruR verif anti (Table 3). Excision of FRT-flanked kan was mediated by transformation with the FLP recombinase-expressing plasmid pCP20 as described in the literature (Datsenko & Wanner, 2000).

### Example 4

### Phage-Based Cre-lox Recombination

To integrate the alginate metabolic pathway (FIG. 4) into the host genome via Cre-lox recombination, the pathway was delivered via phage transduction. A schematic demonstrating the process is shown in Step 3 of FIG. 1.

BAL1075 (Table 2) was transformed with pALG2.3.4 (Table 4) and used for the preparation of lysates from the P1vir bacteriophage (Miller, 1992; Thomason et al., 2007). These lysates were subsequently used to infect an overnight culture of BAL1075 ldhA::loxP-cat-lox5171 pJW168 grown at 30° C. in LB medium with 100 μg/mL ampicillin and 1 mM IPTG. Following 1-hour infection, cells were plated on LB-agar plates with 12.5 μg/mL kanamycin to isolate single colonies. After overnight growth at 37° C., individual colonies were streaked out on LB-kanamycin and LB-chloramphenicol (25 μg/mL) to identify chloramphenicol-sensitive colonies. These colonies were additionally verified for proper end integration by colony PCR using the primer pairs listed above. Correct colonies were also streaked out on LB-ampicillin plates to verify loss Of pJW168. Excision of FRT-flanked kan was mediated by transformation with the FLP recombinase-expressing plasmid pCP20 as described in the literature (Datsenko & Wanner, 2000).

### Example 5

### Effect of Insert Size on Integration Efficiency

Homologous recombination-based methods for genomic integration often exhibit very low efficiencies, particularly for longer cassette lengths. A recent evaluation of a popular λ-RED recombination (Datsenko & Wanner, 2000) revealed that the number of recombinant strains often drops to 0 for insert sizes at or greater than 2.5 kb (Kuhlman & Cox, 2010). To this end, several versions of the donor plasmid which contained partial or complete alginate pathways ranging from 6 kb to 58 kb in length (pALG2.3.4-pALG2.3.8, pALG2.5.4, pALG7.8.4; Table 4) were used to integrate the pathway into the host genome using the protocols described in the Examples above. Efficiencies of integration were then calculated based on the percent of chloramphenicol-sensitive colonies recovered for each recombination protocol (out of a total of about 30 colonies). In addition, two random chloramphenicol-sensitive colonies from each culture were chosen for colony PCR verification. Triplicate lysate preparation and infection experiments were performed for each plasmid version.

The results are shown in FIG. 2 (c). No clear dependence on length was found for the phage-based delivery method, and only a slight drop-off in efficiency was observed for the plasmid-based method. Although the largest fragment tested was 58 kb, phage delivery can potentially mediate the insertion of pieces up to 100 kb in size, a limit imposed by the amount of DNA packaged into the head of a bacteriophage particle (Thomason et al., 2007). The plasmid-based method can likely handle even larger pieces, as long as the genetic fragment can be stably maintained on a single-copy plasmid.

The efficiency studies revealed a two- to four-fold higher rate of integration for the plasmid-based method [FIG. 2 (c)], a phenomenon which likely stems from the more facile recombination of a genetic fragment already present (and stably maintained) within each cell. In stark contrast, successful integration via phage delivery requires that the complete genetic fragment first be transduced into the recipient cell and then properly recombined into the bacterial chromosome. Despite its lower efficiency, however, the transduction protocol remains advantageous for systems in which the fragment for integration resides on another chromosome or within a multi-copy plasmid. The plasmid-based method is ineffective for the latter scenario, as antibiotic resistance can still be imparted by plasmid copies not residing within the genome.

The system demonstrated above is a simple and versatile system for integrating large pieces of DNA into the bacterial chromosome. After the construction of a compatible vector (containing lox sites flanking the region of interest), the entire procedure from start to verification can be completed in under a week.

### Example 6

### Alginate Growth Assays

Fragment integrity of the strains obtained by the Cre-lox recombination process was tested via growth on 2% degraded alginate medium.

Strains used for alginate growth assays were first grown overnight in LB medium at 30° C. One hundred μL cell culture was washed and resuspended in an equal volume of 2% degraded alginate medium (M9 minimal medium with 2% alginate pre-degraded with 10 μg/mL alginate lyase (Sigma, St. Louis, Mo.) overnight at 30° C.), and 4 μL was used to inoculate 196 μL of 2% degraded alginate medium. Alginate growth assays were performed at 30° C. and cell density (OD₆₀₀) was monitored with a BioTek Synergy HT Multidetection microplate reader (BioTek Instruments, Inc., Winooski, Vt.). All liquid cultivations were conducted with at least three biological replicates.

As shown in FIG. 2 (b), 5 individual colonies exhibited identical growth profiles on 2% degraded alginate medium, indicating a high degree of consistency and a low rate of mutation within the genetic fragment. In fact, mutation rates were expected to be much lower than for other integration methods, since the fragments were taken directly from either the donor plasmid or the transduced genetic material, and no PCR amplification steps were required.

### Example 7

### Effect of Gene Copy Number and Chromosomal Location on Growth of Strains Modified Through P1vir Phage Transduction

The recombination system opened several new realms for the metabolic engineering of long, heterologous pathways, making it possible to readily investigate locus-dependent effects (Sousa et al., 1997) and, when used in conjunction with phage transduction, variations due to chromosomal copy number.

Transfer of ldhA::loxP-ALG2.3.4-kan-lox5171 into BAL1075 int(gidB-atpI)::loxP-ALG2.3.4-lox5171 was mediated by P1vir phage transduction (Miller, 1992; Thomason et al., 2007). Proper integration was verified by colony PCR as described above. Excision of FRT-flanked kan was mediated by transformation with the FLP recombinase-expressing plasmid pCP20 as described in the literature (Datsenko & Wanner, 2000). Growth of the strains on 2% degraded alginate medium was then tested as per the protocol described in Example 6.

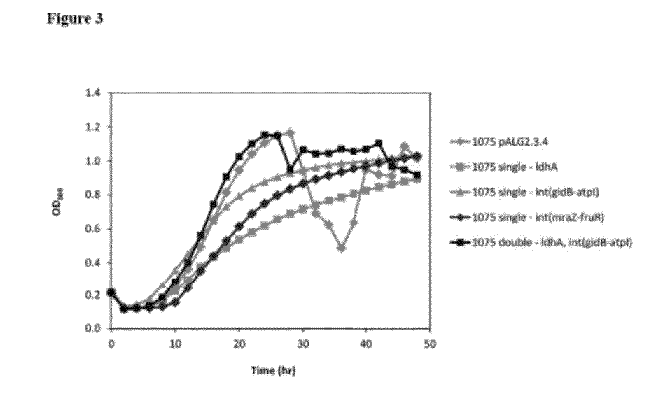

Integration of the alginate metabolic pathway at three separate chromosomal locations led to different growth profiles, presumably due to variations in effective copy number (Sousa et al., 1997). As seen in FIG. 3, integrations occurring at positions closer to the chromosomal origin (oriC) exhibited better growth rates on degraded alginate medium. Although the growth of these single copy integrations still lagged in comparison to a plasmid-based control, such deficiencies were overcome by integration of a second copy of the alginate pathway by phage transduction (FIG. 3).

### Example 8

### Ethanol Production by Strains with Alternate Backgrounds

The alginate metabolic pathway (FIG. 4) was integrated into alternate strain backgrounds, which were then tested for production of ethanol.

Transfer of int(gidB-atpI)::loxP-ALG2.3.4-lox5171 and int(mraZ-fruR)::loxP-ALG2.3.4-lox5171 into alternate strain backgrounds (BAL1301, BAL1304, BAL1373, and BAL1450, Table 2) was mediated by P1vir phage transduction (Miller, 1992; Thomason et al., 2007). Proper integration was verified by colony PCR as described above. Excision of FRT-flanked kan was mediated by transformation with the FLP recombinase-expressing plasmid pCP20 as described in the literature (Datsenko & Wanner, 2000).

Ethanol fermentations were conducted in stirred bottles containing 100 mL M9 minimal media with 6% sugars (mannitol:alginate:glucose at a ratio of 1.5:3.75:0.5) and 0.5% LB. Cultures were inoculated to a starting cell density (OD₆₀₀) of ˜0.6, and ethanol concentrations were measured using a quantified by High Performance Liquid Chromatography (Shimadzu, Columbia, Md.) equipped with an organic acid column (Phenomenex, Torrance, Calif.). Chromatography was operated at 60° C. using 5 mM H₂SO₄ as a mobile phase at a flow rate of 1 mL/min (5 μL injection volume, 15 minute isocratic method). Ethanol peaks were detected using a refractive index detector and compared to chemical standards.

Integrated strains expressing a secreted alginate lyase system (N455+SM0524), which is able to degrade alginate polymer into smaller oligomers, exhibited increases in ethanol titers and productivities compared to a plasmid-based control (Table 6). Indeed, ethanol production in these integrated strains exceeded the control by up to 72% after 24 hours and up to 10.7% after 40 hours. In summary, this method successfully circumvented inherent plasmid instabilities and led to the construction of genetically robust strains of E. coli capable of growing and producing ethanol at significantly enhanced productivities and titers from alginate-containing media.

Table 6 shows ethanol production of integrated strains in alginate-containing media.

| | # Inte- | Ethanol Concen- | | % increase | |
|---|---|---|---|---|---|
| | grated | tration (g/L) | | above 847 | |
| Strain Background | Copies | 24 hour | 40 hour | 24 hour | 40 hour |
| 847 N455+SM0524 | Plasmid | 6.7 | 13.4 | — | — |
| 1301 N455+SM0524 | 1 | 10.5 | 14.1 | 58.1 | 5.2 |
| 1302 N455+SM0524 | 1 | 8.7 | 14.0 | 30.0 | 4.0 |
| 1303 N455+SM0524 | 1 | — | 14.9 | — | 10.7 |
| 1304 N455+SM0524 | 1 | — | 14.1 | — | 4.8 |
| 1301 N455+SM0524 | 2 | 11.5 | 14.7 | 72.0 | 9.3 |
| 1303 N455+SM0524 | 2 | 10.2 | 14.2 | 52.6 | 5.5 |

Ethanol fermentations were also conducted with 5% sugars, utilizing the same methods described above. Integrated strains expressing a secreted alginate lyase system (N455+SM0524), which is able to degrade alginate polymer into smaller oligomers, exhibited increases in ethanol titers and productivities compared to a plasmid-based control (Table 7). The genotype of BAL1810, BAL1373, and BAL1450 are described in Table 2. The strains were generated as described in Examples 1-3 above.

Table 7 shows ethanol titers and productivities of strains in alginate-containing media

| | | | Ethanol | % increase | Ethanol | % increase |
|---|---|---|---|---|---|---|
| | Ethanol | Alginate | Titer (g/L) - | above BAL | Productivity | above BAL |
| Strain # | pathway | pathway | 66 hr | 1810 | (g/L-hr) | 1810 |
| BAL1810 | plasmid | plasmid | 11.8 | — | 0.13 | — |
| BAL1373 | integrated | plasmid | 14.9 | 26% | 0.17 | 31% |
| BAL1450 | integrated | integrated | 18.8 | 59% | 0.24 | 85% |

### REFERENCES

1. Abremski, K., Hoess, R., & Sternberg N. Studies on the properties of P1 site-specific recombination: evidence for topologically unlinked products following recombination. Cell 32(4), 1301-11 (1983).
2. Birling, M. C., Gofflot, F., & Warot X. Site-specific recombinases for manipulation of the mouse genome. Methods Mol Bio, 561, 245-263 (2009).
3. Birnbaum, S. & Bailey, J. E. Plasmid presence changes the relative levels of many host cell proteins and ribosome components in recombinant Escherichia coli. Biotechnol Bioeng 37, 736-745 (1991).
4. Cox, M. M. The FLP protein of the yeast 2-eum plasmid: Expression of a eukaryotic genetic recombination system in Escherichia coli. Proc Natl Acad Sci USA 80, 4223-4227 (1983).
5. Datsenko, K. A. & Wanner, B. L. One-step inactivation of chromosomal genes in Escherichia coli K-12 using PCR products. Proc Natl Acad Sci USA 97, 6640-6645 (2000).
6. Hardy, S., Kitamura, M., Harris-Stansil, T., Dai, Y., & Phipps, M. L. Construction of adenovirus vectors through Cre-lox recombination. J Virol 71(3):1842-9 (1997).
7. Jones, K. L., Kim, S. W. & Keasling, J. D. Low-copy plasmids can perform as well as or better than high-copy plasmids for metabolic engineering of bacteria. Metab Eng 2, 328-338 (2000).
8. Koning, S. M., Elferink, M. G., Konings, W. N., & Driessen, A. J. Cellobiose uptake in the hyperthermophilic archaeon Pyrococcus furiosus is mediated by an inducible, high-affinity ABC transporter. J Bacteriol 183(17), 4979-4984 (2001).
9. Kuhlman, T. E. & Cox, E. C. Site-specific chromosomal integration of large synthetic constructs. Nucleic Acids Res 38, e92 (2010).
10. Lee, G. & Saito, I. Role of nucleotide sequences of loxP spacer region in Cre-mediated recombination. Gene 216, 55-65 (1998).
11. Luo, H. & Kausch, A. P. Application of FLP/FRT site-specific DNA recombination system in plants. Genetic Eng (NY) 24, 1-16 (2002).
12. McLeod, M., Craft, S., & Broach, J. R. Identification of the crossover site during FLP-mediated recombination in the Saccharomyces cerevisiae plasmid 2 microns circle. Mol Cell Bio 6, 3357-3367 (1986).
13. Miller, J. H. A short course in bacterial genetics: a laboratory manual and handbook for Escherichia coli and related bacteria. (Cold Spring Harbor Laboratory Press, Plainview, N.Y.; 1992).
14. Schlosser, A., Jantos, J., Hackmann, K., & Schrempf H. Characterization of the binding protein-dependent cellobiose and cellotriose transport system of the cellulose degrader Streptomyces reticuli. Appl Environ Microbiol 65(6), 2636-2643 (1999).
15. Schweizer, H. P. Applications of the Saccharomyces cerevisiae Flp-FRT system in bacterial genetics. J Mol Microbiol Biotechnol 5, 67-77 (2003).
16. Sousa, C., de Lorenzo, V. & Cebolla, A. Modulation of gene expression through chromosomal positioning in Escherichia coli. Microbiology 143 (Pt 6), 2071-2078 (1997).
17. Sternberg, N., Hamilton, D., Austin, S., Yarmolinsky, M., & Hoess, R. Site-specific Recombination and Its Role in the Life Cycle of Bacteriophage P1. Cold Spring Harbor Symp. Quant. Biol. 45, 297-309 (Cold Spring Harbor Laboratory Press, Cold Spring Harbor, N.Y. (1981)).
18. Thomason, L. C., Costantino, N. & Court, D. L. E. coli genome manipulation by P1 transduction. Curr Protoc Mol Biol Chapter 1, Unit 117 (2007).
19. Yu, D., Ellis, H. M., Lee, E. C., Jenkins, N. A., Copeland, N. G., & Court, D. L. An efficient recombination system for chromosome engineering in Escherichia coli. Proc Natl Acad Sci USA 97, 5978-5983 (2000).
20. Yu, D., Sawitzke, J. A., Ellis, H. & Court, D. L. Recombineering with overlapping single-stranded DNA oligonucleotides: testing a recombination intermediate. Proc Natl Acad Sci USA 100, 7207-7212 (2003).

What is claimed is:

1. An E. coli strain comprising a recombinant polynucleotide wherein the E. coli strain comprises a genome wherein the recombinant polynucleotide is stably integrated into the genome and wherein the recombinant polynucleotide comprises a nucleotide sequence encoding an alginate lyase, a DEHU reductase, and an alginate transporter and wherein integration of the recombinant polynucleotide into the genome modifies said E. coli strain to be able to grow on alginate-containing or alginate-derived media.

2. An E. coli strain comprising a recombinant polynucleotide wherein the E. coli strain comprises a genome wherein the recombinant polynucleotide is stably integrated into the genome, is at least 11 kilobases in size, and comprises a nucleotide sequence encoding one or more heterologous genes.

3. The E. coli strain of claim 2, wherein the size of the recombinant polynucleotide is selected from the group consisting of:
A) at least 12 kilobases;
B) at least 13 kilobases; and
C) at least 14 kilobases.

4. The E. coli strain of claim 2, wherein said one or more heterologous genes integrated into the genome is an alginate lyase, a DEHU reductase, and/or an alginate transporter and wherein integration of the one or more heterologous genes into the genome modifies said E. coli strain to be able to grow on alginate-containing or alginate-derived media.

5. The E. coli strain of claim 2, wherein said one or more heterologous genes integrated into the genome is an endo-type cellulase, an exo-type cellulase, a β-glucosidase, and/or a cellulose/cellobiose transporter and wherein integration of the one or more heterologous genes into the genome modifies said E. coli strain to be able to grow on cellulose/cellobiose-containing media.

6. The E. coli strain of claim 1, wherein said recombinant polynucleotide is positioned between two lox sites in said genome.

7. A method of integrating a recombinant polynucleotide in the genome of an E. coli strain comprising:
A) providing an E. coli strain comprising a genome having a first lox site and a second lox site integrated in said genome of the E. coli strain, wherein the first lox site comprises a different sequence from the second lox site such that the first and second lox sites are incapable of recombining with each other;
B) transforming said E. coli strain, with a first plasmid and a second plasmid, wherein the first plasmid comprises a recombinant polynucleotide comprising a nucleotide sequence encoding one or more heterologous genes, wherein said recombinant polynucleotide is bounded by a third lox site and a fourth lox site wherein the third lox site has the same sequence as the first lox site and the fourth lox site has the same sequence as the second lox site, and wherein the second plasmid encodes Cre recombinase; and
C) culturing said E. coli strain under conditions such that Cre recombinase is expressed, wherein Cre recombinase expression results in homologous recombination between the first and third lox sites and between the second and fourth lox sites and integration of the recombinant polynucleotide into the genome of the E. coli strain in between the first and second lox sites.

8. A method of integrating a recombinant polynucleotide in the genome of an E. coli strain comprising:
A) providing an E. coli strain comprising a genome having a first lox site and a second lox site integrated in said genome of the E. coli strain, wherein the first lox site comprises a different sequence from the second lox site such that the first and second lox sites are incapable of recombining with each other, and comprising a plasmid encoding Cre recombinase;
B) providing a donor cell comprising recombinant polynucleotide, said recombinant polynucleotide comprising a nucleotide sequence encoding one or more heterologous genes, wherein said recombinant polynucleotide is bounded by a third lox site and a fourth lox site wherein the third lox site has the same sequence as the first lox site and the fourth lox site has the same sequence as the second lox site;
C) infecting the donor cell with a phage such that phage particles comprising said recombinant polynucleotide are produced and released from the donor cell;
D) culturing said E. coli strain such that Cre recombinase is expressed; and
E) infecting said E. coli strain expressing Cre recombinase with the phage particles, wherein Cre recombinase expression results in homologous recombination between the first and third lox sites and between the second and fourth lox sites and integration of the recombinant polynucleotide into the genome of the E. coli strain in between the first and second lox sites.

9. The method of claim 7, further comprising:
D) growing said E. coli strain in media and under conditions wherein said one or more heterologous genes are expressed and a commodity chemical is produced; and
E) collecting said commodity chemical.

10. The method of claim 8, further comprising:
F) growing said E. coli strain in media and under conditions wherein said one or more heterologous genes are expressed and a commodity chemical is produced; and
G) collecting said commodity chemical.

11. The method of claim 7, wherein said one or more heterologous genes integrated into the genome are an alginate lyase, a DEHU reductase, and/or an alginate transporter and wherein integration of the one or more heterologous genes into the genome modifies said E. coli strain to be able to grow on alginate-containing or alginate-derived media.

12. The method of claim 7, wherein said one or more heterologous genes integrated into the genome are an alginate lyase, a DEHU reductase, and an alginate transporter and wherein integration of the heterologous genes into the genome modifies said E. coli strain to be able to grow on alginate-containing or alginate-derived media.

13. The method of claim 7, wherein said one or more heterologous genes integrated into the genome are an endo-type cellulase, an exo-type cellulase, a β-glucosidase, and/or a cellulose/cellobiose transporter and wherein integration of the one or more heterologous genes into the genome modifies said E. coli strain to be able to grow on cellulose/cellobiose-containing media.

14. The method of claim 8, wherein the phage is P1vir.

15. The method of claim 8, wherein the size of the recombinant polynucleotide is at least 11 kilobases.

16. The method of claim 8, wherein said one or more heterologous genes integrated into the genome are an alginate lyase, a DEHU reductase, and/or an alginate transporter and wherein integration of the one or more heterologous genes into the genome modifies said E. coli strain to be able to grow on alginate-containing or alginate-derived media.

17. The method of claim 8, wherein said one or more heterologous genes integrated into the genome are an alginate lyase, a DEHU reductase, and an alginate transporter and wherein integration of the heterologous genes into the genome modifies said E. coli strain to be able to grow on alginate-containing or alginate-derived media.

18. The method of claim 8, wherein said one or more heterologous genes integrated into the genome are an endo-type cellulase, an exo-type cellulase, a β-glucosidase, and/or a cellulose/cellobiose transporter and wherein integration of the one or more heterologous genes into the genome modifies said E. coli strain to be able to grow on cellulose/cellobiose-containing media.

19. The method of claim 9, wherein said commodity chemical is ethanol.

20. The method of claim 10, wherein said commodity chemical is ethanol.


SEQUENCE LISTING (35 sequences)

SEQ ID NO: 1 - DNA, 88 bp, Artificial Sequence
  Synthetic Construct
ccagattgcttaagttttgcagcgtagtcataacttcgtatagtacacattatacgaagttattcggcacgtaagaggttccaacttt

SEQ ID NO: 2 - DNA, 87 bp, Artificial Sequence
  Synthetic Construct
tacgacaagaagtacctgcaacaggtgaataacttcgtataatgtatgctatacgaagttatggcgtttaagggcaccaataactgc

SEQ ID NO: 3 - DNA, 89 bp, Artificial Sequence
  Synthetic Construct
cctgggttgcaggggagcggcaagattaaaccagttcgttcgggcaggtttcgcctttttccagattgcttaagttttgcagcgtagtc

SEQ ID NO: 4 - DNA, 88 bp, Artificial Sequence
  Synthetic Construct
atgtgattcaacatcactggagaaagtcttatgaaactcgccgtttatagcacaaaacagtacgacaagaagtacctgcaacaggtga

SEQ ID NO: 5 - DNA, 30 bp, Artificial Sequence
  Synthetic Construct
gcatgggtagttaatatcctgatttagcga

SEQ ID NO: 6 - DNA, 28 bp, Artificial Sequence
  Synthetic Construct
gaaaggtcattggggattatctgaatca

SEQ ID NO: 7 - DNA, 25 bp, Artificial Sequence
  Synthetic Construct
ctgaccgttctgtccgtcacttccc

SEQ ID NO: 8 - DNA, 85 bp, Artificial Sequence
  Synthetic Construct
cttaatcgctggcttttcttctttcaaatcaattcatttaaataagagccgagtacttaaggcgtttaagggcaccaataactgc

SEQ ID NO: 9 - DNA, 90 bp, Artificial Sequence
  Synthetic Construct
ccctgggccaacttttggcgaaaatgagacgttgaataacttcgtatagtacacattatacgaagttatatccgtcgacctgcagttcga

SEQ ID NO: 10 - DNA, 80 bp, Artificial Sequence
  Synthetic Construct
tgacggaagatcacttcgcagaataaataaatcctggtgtccctgttgataccgggaagccctgggccaacttttggcga

SEQ ID NO: 11 - DNA, 83 bp, Artificial Sequence
  Synthetic Construct
cttaatcgctggcttttcttctttcaaatcaattcatttaaataagagccgagtacttaatgtgtaggctggagctgcttcga

SEQ ID NO: 12 - DNA, 83 bp, Artificial Sequence
  Synthetic Construct
caatgaactctttctttatcctagatgaaaatccatgggaagaacttggtggcggcattatgtgtaggctggagctgcttcga

SEQ ID NO: 13 - DNA, 83 bp, Artificial Sequence
  Synthetic Construct
gatttagaatactgagaggtgaaaaatcccggctgtcgcataactacttgtcaggtacagtgtgtaggctggagctgcttcga

SEQ ID NO: 14 - DNA, 83 bp, Artificial Sequence
  Synthetic Construct
caagaacaacgcagaaaagccactctaaactcgacagttattgagtggccttcagatcaatgtgtaggctggagctgcttcga

SEQ ID NO: 15 - DNA, 83 bp, Artificial Sequence
  Synthetic Construct
actctagtgctaattgtcattctgtctttctactgatccagccctctcaaagcctgtatctgtgtaggctggagctgcttcga

SEQ ID NO: 16 - DNA, 60 bp, Artificial Sequence
  Synthetic Construct
ggccagtgaattgtaatacgactcactatagggcgaatgtgtaggctggagctgcttcga

SEQ ID NO: 17 - DNA, 90 bp, Artificial Sequence
  Synthetic Construct
attaatgtgaccttggtatcaatgagggtgtacgtataacttcgtatagcatacattatacgaagttatatccgtcgacctgcagttcga

SEQ ID NO: 18 - DNA, 60 bp, Artificial Sequence
  Synthetic Construct
tttcccagtcacgacgttgtaaaacgacggccagtgaattgtaatacgactcactatagg

SEQ ID NO: 19 - DNA, 60 bp, Artificial Sequence
  Synthetic Construct
gttttatatgagttttaagatgaacttggtattaatgtgaccttggtatcaatgagggtg

SEQ ID NO: 20 - DNA, 90 bp, Artificial Sequence
  Synthetic Construct
gatgcctttgcaagtttatgatatttcagtcataacttcgtatagtacacattatacgaagttattcggcacgtaagaggttccaacttt

SEQ ID NO: 21 - DNA, 89 bp, Artificial Sequence
  Synthetic Construct
tgttcactcttttgcatcaacaagataacgataacttcgtataatgtatgctatacgaagttatggcgtttaagggcaccaataactgc

SEQ ID NO: 22 - DNA, 60 bp, Artificial Sequence
  Synthetic Construct
cgcacagcatatttatttacttggcaaatgatgcctttgcaagtttatgatatttcagtc

SEQ ID NO: 23 - DNA, 60 bp, Artificial Sequence
  Synthetic Construct
tactgatataactggttacatttaacgccatgttcactcttttgcatcaacaagataacg

SEQ ID NO: 24 - DNA, 29 bp, Artificial Sequence
  Synthetic Construct
ggtcgaatcagtggttaaacttcaggttc

SEQ ID NO: 25 - DNA, 24 bp, Artificial Sequence
  Synthetic Construct
gtttcgactcacgagcgacacaga

SEQ ID NO: 26 - DNA, 90 bp, Artificial Sequence
  Synthetic Construct
ggttaacagtccctgttgcgtctgtgtggcgataacttcgtatagtacacattatacgaagttattcggcacgtaagaggttccaacttt

SEQ ID NO: 27 - DNA, 90 bp, Artificial Sequence
  Synthetic Construct
cgggactggacatcaatatgcttaaagtaaaataacttcgtataatgtatgctatacgaagttatggcgtttaagggcaccaataactgc

SEQ ID NO: 28 - DNA, 60 bp, Artificial Sequence
  Synthetic Construct
cgacgcgagcggcattttaggacatatcttccccggttaacagtccctgttgcgtctgtg

SEQ ID NO: 29 - DNA, 60 bp, Artificial Sequence
  Synthetic Construct
cgccaggtgaatttccctctggcgcgtagagtacgggactggacatcaatatgcttaaag

SEQ ID NO: 30 - DNA, 21 bp, Artificial Sequence
  Synthetic Construct
gtgtcagtttgcgacgcgagc

SEQ ID NO: 31 - DNA, 25 bp, Artificial Sequence
  Synthetic Construct
gcgtaagccaaaacctggtttaacg

SEQ ID NO: 32 - DNA, 83 bp, Artificial Sequence
  Synthetic Construct
ttaattgcgttgcgctcactgcccgctttccagtcgggaaacctgtcgtgccagctgcattgtgtaggctggagctgcttcga

SEQ ID NO: 33 - DNA, 89 bp, Artificial Sequence
  Synthetic Construct
cgttcatttcacttcatttgccgcatctcacattataacttcgtatagcatacattatacgaagttatatccgtcgacctgcagttcga

SEQ ID NO: 34 - DNA, 60 bp, Artificial Sequence
  Synthetic Construct
ttctgcgttgttcttgagtctaactctacgtaatatccgttcatttcacttcatttgccg

SEQ ID NO: 35 - DNA, 83 bp, Artificial Sequence
  Synthetic Construct
aataatctaagataattactgtcctaattttctaagacctaaacaaaagccagcttaatctgtgtaggctggagctgcttcga